(19) United States
(12) Patent Application Publication
(10) Pub. No.: US 2026/0199517 A1
(43) Pub. Date: Jul. 16, 2026

(54) LYOPHILIZATION BUFFERS FOR PEPTIDE-DECORATED LIPOSOMES

(71) Applicant: Haima Therapeutics, LLC, Cleveland, OH (US)

(72) Inventors: Andrew Ditto, Hudson, OH (US); Shrijal Desai, Lake Villa, IL (US); Christa Pawlowski, Solon, OH (US)

(21) Appl. No.: 19/130,805

(22) PCT Filed: Nov. 17, 2023
(86) PCT No.: PCT/US23/80229

§ 371 Date: May 16, 2025

Related U.S. Application Data
(60) Provisional application No. 63/426,530, filed on Nov. 18, 2022.

(51) Int. Cl. A61K 47/69; A61K 9/51; A61K 47/02; A61K 47/20; A61K 47/22; A61K 47/26; A61K 47/40; A61K 47/64; A61P 7/04

(52) U.S. Cl. CPC A61K 47/6929; A61K 9/5123; A61K 47/02; A61K 47/20; A61K 47/22; A61K 47/26; A61K 47/40; A61K 47/64; A61K 47/6911; A61P 7/04

(57) ABSTRACT

Methods and compositions are provided for lyophilizing compositions comprising a peptide-decorated lipid.

### RELATED APPLICATIONS

This application claims the benefit of priority to U.S. Provisional Patent Application Ser. No. 63/426,530, filed on Nov. 18, 2022, which is hereby incorporated by reference in its entirety.

### GOVERNMENT SUPPORT

This invention was made with government support under Grant Number 1951301, awarded by the National Science Foundation and Grant Number W81XWH2010628 awarded by the Department of Defense. The government has certain rights in the invention.

### BACKGROUND

Some biomolecules, including therapeutic compositions, reagents, and the like, have a limited shelf-life. Much care, usually at great expense, is required to store these compositions such that they retain their desired biological activity when used after storage. Certain storage conditions such as temperature and pH can be optimized to prolong the shelf life of the biomolecule, but long-term storage may still be unattainable due to storage conditions that result in structural modifications that render the biomolecule inactive. Accordingly, there is a need for storage conditions for peptide-decorated lipids (e.g., liposomes). Embodiments of the present invention are directed to this and other important needs.

### SUMMARY

The present disclosure is based, at least in part, on the discovery that a lyophilization buffer comprising the lyoprotectant cyclodextrin or its derivatives maintains the size range and functionality of lyophilized lipid particles decorated with binding peptides after reconstitution.

In certain embodiments, the disclosure relates to a composition comprising a plurality of lipid particles and a liquid component, wherein the liquid component comprises water and a cyclodextrin at a concentration from about 1% (w/v) to about 20% (w/v) of the liquid component, wherein each lipid particle comprises a lipid and a peptide conjugate, wherein the peptide of the peptide conjugate is associated with the external surface of the lipid particle. In certain embodiments, the disclosure relates to a composition comprising a plurality of lipid particles and a liquid component, wherein the liquid component comprises water and a lyoprotectant at a concentration from about 1% (w/v) to about 20% (w/v) of the liquid component, wherein each lipid particle comprises a lipid and a plurality of peptide conjugates; the peptide conjugates are selected from platelet binding peptide (PBP) conjugates, von Willebrand factor-binding peptide (VBP) conjugates, and collagen-binding peptide (CBP) conjugates, or a combination thereof, wherein the PBP conjugate is a fibrinogen mimetic peptide (FMP) conjugate or a P-selectin binding peptide conjugate; and the plurality of PBP conjugates, VBP conjugates, and/or CBP conjugates are conjugated to an outer surface of the particle, wherein the PBP conjugates, VBP conjugates, and CBP conjugates, taken together, are present at <5 molar percent of the particle.

In certain embodiments, the disclosure relates to a method of forming a dried composition, comprising:
incubating any of the compositions described herein at a temperature from about 0 to about −210° C. to form a cooled composition; and
incubating the cooled composition at a temperature from about 0 to about −60° C. at a pressure from about 0.1 to about 1.0 mTorr, thereby forming a dried composition.

In certain embodiments, the disclosure relates to a composition made by any of the methods described herein.

In certain embodiments, the disclosure relates to a method of forming a reconstituted composition comprising contacting any of the compositions described herein with water, for example, for less than about 30 seconds, thereby forming the reconstituted composition.

In certain embodiments, the disclosure relates to a reconstituted composition made by any of the methods described herein.

In certain embodiments, the disclosure relates to a method of diminishing bleeding, treating hemorrhage, treating a vascular injury, promoting hemostasis, preventing or inhibiting platelet aggregation, promoting aggregation of activated platelets on a site with exposed vWF and collagen, or treating cancer in a subject in need thereof, comprising administering to the subject an effective amount of any of the reconstituted compositions described herein.

### BRIEF DESCRIPTION OF THE DRAWINGS

FIGS. 1A-1AA show the size range and lyophilized cake images for lyophilized synthetic platelets (Post-Lyo, grey) and pre-lyophilized synthetic platelets (Pre-Lyo, black) in various buffers containing the buffering agents Tris or HEPES and lyoprotectants sucrose, dextrose, or hydroxypropyl-beta-cyclodextrin (HP-3-CD). FIG. 1A is an intensity diameter histogram showing the relative frequencies of different intensity diameters for synthetic platelets pre and post-lyophilization in a 10% dextrose with Tris (3 mg/mL) lyophilization buffer. FIG. 1B is an image of the lyophilization cake of synthetic platelets lyophilized in a 10% dextrose with Tris (3 mg/mL) lyophilization buffer. FIG. 1C is an intensity diameter histogram showing the relative frequencies of different intensity diameters for synthetic platelets pre and post-lyophilization in a 10% dextrose with Tris (3 mg/mL) lyophilization buffer. FIG. 1D is an intensity diameter histogram showing the relative frequencies of different intensity diameters for synthetic platelets pre and post-lyophilization in a 10% dextrose with HEPES (2 mg/mL) lyophilization buffer. FIG. 1E is an image of the lyophilization cake of synthetic platelets lyophilized in a 10% dextrose with HEPES (2 mg/mL) lyophilization buffer. FIG. 1F is an intensity diameter histogram showing the relative frequencies of different intensity diameters for synthetic platelets pre and post-lyophilization in a 10% dextrose with HEPES (2 mg/mL) lyophilization buffer. FIG. 1G is an image of the lyophilization cake of synthetic platelets lyophilized in a 10% dextrose with HEPES (2 mg/mL) lyophilization buffer. FIG. 1H is an intensity diameter histogram showing the relative frequencies of different intensity diameters for synthetic platelets pre and post-lyophilization in a 10% HP-β-CD with Tris (3 mg/mL) lyophilization buffer. FIG. 1I is an image of the lyophilization cake of synthetic platelets lyophilized in a 100% HP-β-CD with Tris (3 mg/mL) lyophilization buffer. FIG. 1J is an intensity diameter histogram showing the relative frequencies of different intensity diameters for synthetic platelets pre and post-lyophilization in a 10% HP-β-CD with Tris (3 mg/mL) lyophilization buffer. FIG. 1K is an image of the lyophilization cake of synthetic platelets lyophilized in a 10% HP-β-CD with Tris (3 mg/mL) lyophilization buffer. FIG. 1L is an intensity diameter histogram showing the relative frequencies of different intensity diameters for synthetic platelets pre and post-lyophilization in a 10% HP-β-CD with HEPES (2 mg/mL) lyophilization buffer. FIG. 1M is an image of the lyophilization cake of synthetic platelets lyophilized in a 10% HP-β-CD with HEPES (2 mg/mL) lyophilization buffer. FIG. 1N is an intensity diameter histogram showing the relative frequencies of different intensity diameters for synthetic platelets pre and post-lyophilization in a 10% HP-β-CD with HEPES (2 mg/mL) lyophilization buffer. FIG. 1O is an image of the lyophilization cake of synthetic platelets lyophilized in a 10% HP-β-CD with HEPES (2 mg/mL) lyophilization buffer. FIG. 1P is an intensity diameter histogram showing the relative frequencies of different intensity diameters for synthetic platelets pre and post-lyophilization in a 5% sucrose with Tris (3 mg/mL) lyophilization buffer. FIG. 1Q is an image of the lyophilization cake of synthetic platelets lyophilized in a 5% sucrose with Tris (3 mg/mL) lyophilization buffer. FIG. 1R is an intensity diameter histogram showing the relative frequencies of different intensity diameters for synthetic platelets pre and post-lyophilization in a 5% sucrose with Tris (3 mg/mL) lyophilization buffer. FIG. 1S is an image of the lyophilization cake of synthetic platelets lyophilized in a 5% sucrose with Tris (3 mg/mL) lyophilization buffer. FIG. 1T is an intensity diameter histogram showing the relative frequencies of different intensity diameters for synthetic platelets pre and post-lyophilization in a 10% sucrose with HEPES (2 mg/mL) lyophilization buffer. FIG. 1U is an image of the lyophilization cake of synthetic platelets lyophilized in a 10% sucrose with HEPES (2 mg/mL) lyophilization buffer. FIG. 1V is an intensity diameter histogram showing the relative frequencies of different intensity diameters for synthetic platelets pre and post-lyophilization in a 10% sucrose with HEPES (2 mg/mL) lyophilization buffer. FIG. 1W is an image of the lyophilization cake of synthetic platelets lyophilized in a 10% sucrose with HEPES (2 mg/mL) lyophilization buffer. FIG. 1X is an intensity diameter histogram showing the relative frequencies of different intensity diameters for synthetic platelets pre and post-lyophilization in a 10% trehalose with HEPES (2 mg/mL) lyophilization buffer. FIG. 1Y is an image of the lyophilization cake of synthetic platelets lyophilized in a 10% trehalose with HEPES (2 mg/mL) lyophilization buffer. FIG. 1Z is an intensity diameter histogram showing the relative frequencies of different intensity diameters for synthetic platelets pre and post-lyophilization in a 10% trehalose with HEPES (2 mg/mL) lyophilization buffer. FIG. 1AA is an image of the lyophilization cake of synthetic platelets lyophilized in a 10% trehalose with HEPES (2 mg/mL) lyophilization buffer.

FIG. 2A, FIG. 2B, FIG. 2C, FIG. 2D, and FIG. 2E show intensity diameter histogram plots for five (5) repeat lots of lyophilized synthetic platelets (Post-Lyo, grey) and pre-lyophilized synthetic platelets (Pre-Lyo, black) with 10% (w/v) HP-β-CD and 2 mg/mL HEPES pH 7.0 Buffer.

FIGS. 3A-3C characterize pre-lyophilized synthetic platelets (Pre-Lyo) and lyophilized synthetic platelets (Post-Lyo). FIG. 3A shows images of cryo-TEM (13,000× magnification) of Pre-Lyo and Post-Lyo synthetic platelets. FIG. 3B is size histograms of Pre-Lyo and Post-Lyo synthetic platelets. FIG. 3C is tables of morphology statistics.

FIG. 4A-4C characterize lyophilized synthetic platelet shelf-life based on physicochemical properties of particle size and zeta potential. FIG. 4A shows intensity hydrodynamic diameter histograms for lyophilized synthetic platelets (Post-lyo) after storage at various temperatures including −20° C., 4° C., ambient (room temperature, RT), and 50° C. for 28, 60, and 90 days and reconstitution (dark grey) compared to pre-lyophilized synthetic platelets (Pre-Lyo) (Note: −20° C. and 50° C. are accelerated conditions, so the final time point was Day 60).

FIG. 4B summarizes hydrodynamic diameter (nm), mean intensity diameter (nm), intensity diameter D90 (nm), PDI, and zeta potential (mV) parameters pre-lyophilization and post-lyophilization and storage at −20° C., 4° C., ambient (room temperature, RT), and 50° C. for 28, 60, and 90 days. FIG. 4C shows mean intensity hydrodynamic diameter values over time (90 days) for lyophilized synthetic platelets stored at −20° C. (black square), 4° C. (black upside-down triangle), ambient (room temperature, RT, black circle), and 50° C. (black triangle). Particle mean intensity diameter stays within +/−25% of pre-Lyo values (dashed horizontal lines).

FIG. 5A is a graph of the percentage of platelets bound to either pre-lyophilized synthetic platelets (Particle1) or post-lyophilized synthetic platelets (Particle1) in either 2 mg/mL HEPES or 3 mg/mL Tris+10% (w/v) HP-β-CD (pH 7), as measured by flow cytometry.

FIG. 5B is a graph of the mean Cy5 fluorescence of platelet-bound pre-lyophilized synthetic platelets (Particle1) or post-lyophilized synthetic platelets (Particle1) in either 2 mg/mL HEPES or 3 mg/mL Tris+10% (w/v) HP-β-CD (pH 7) as measured by flow cytometry.

FIG. 6A is a graph showing mouse tail bleeding time after administration of lipid particles described herein, both before lyophilization and after being reconstituted from a lyophilized state.

FIG. 6B is a graph showing mouse blood loss after administration of lipid particles described herein, both before lyophilization and after being reconstituted from a lyophilized state.

FIG. 7A shows the zeta potential of lyophilized Particle1 after reconstitution.

FIG. 7B shows the mean intensity diameter of lyophilized Particle1 after reconstitution.

FIG. 7C shows the particle concentration of lyophilized Particle1 after reconstitution.

FIG. 7D shows the polydispersity index (PDI) of lyophilized Particle1 after reconstitution.

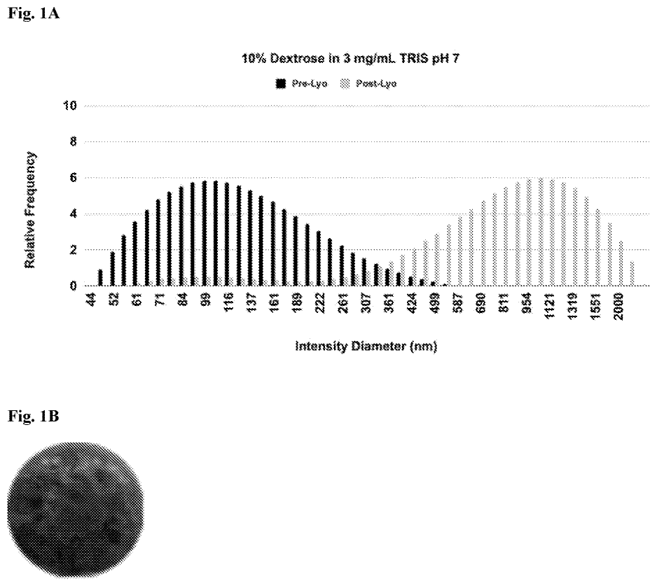

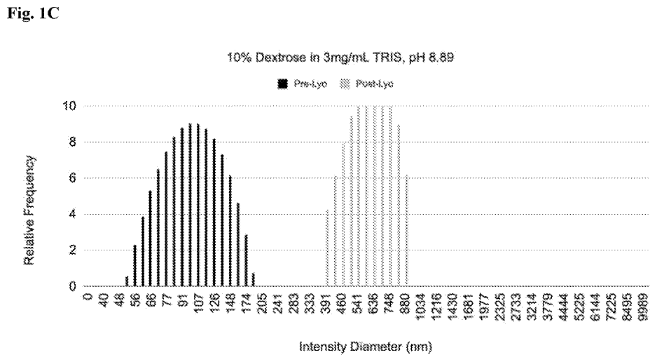

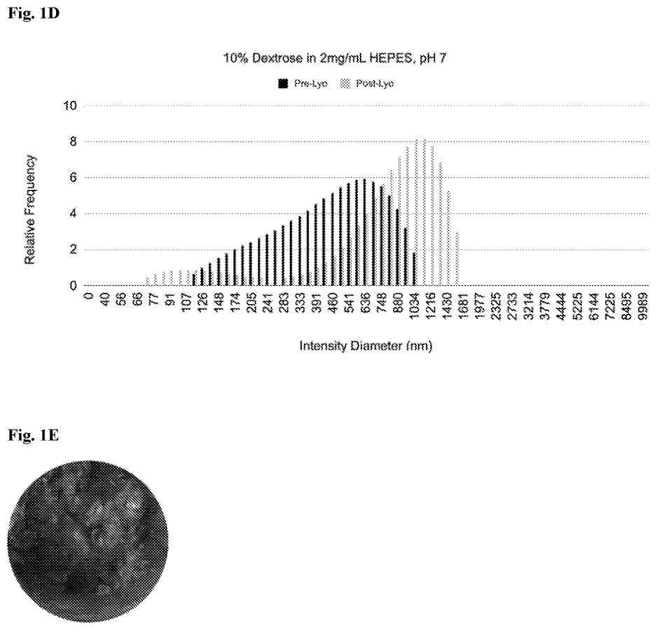

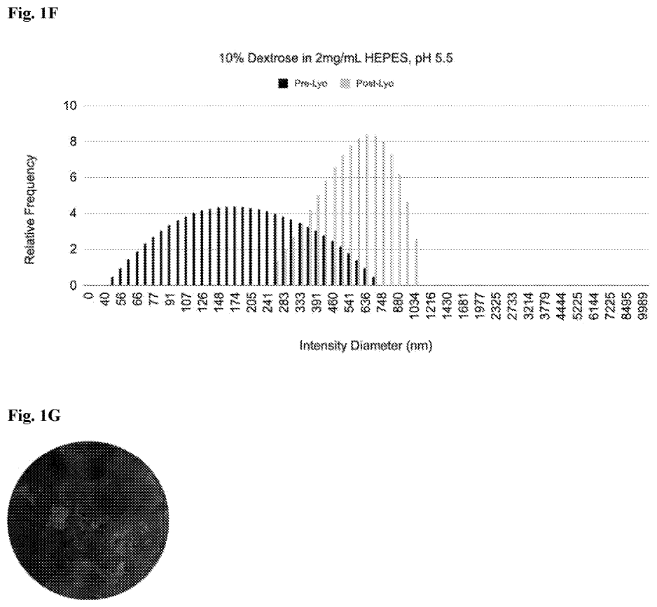

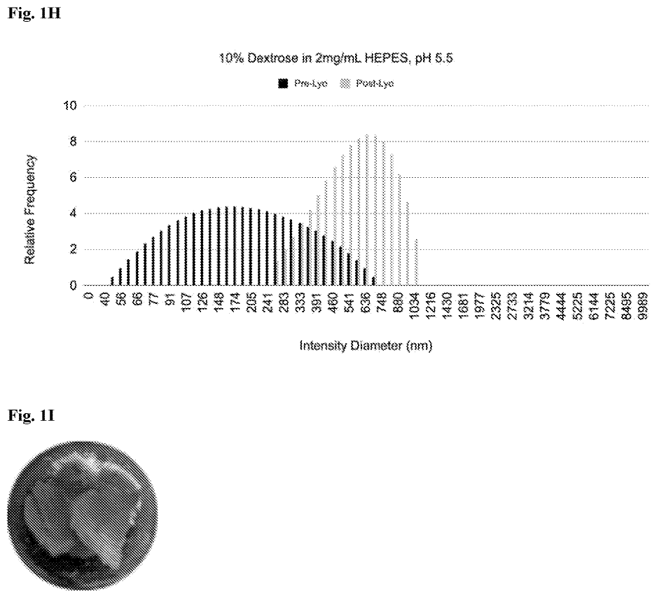

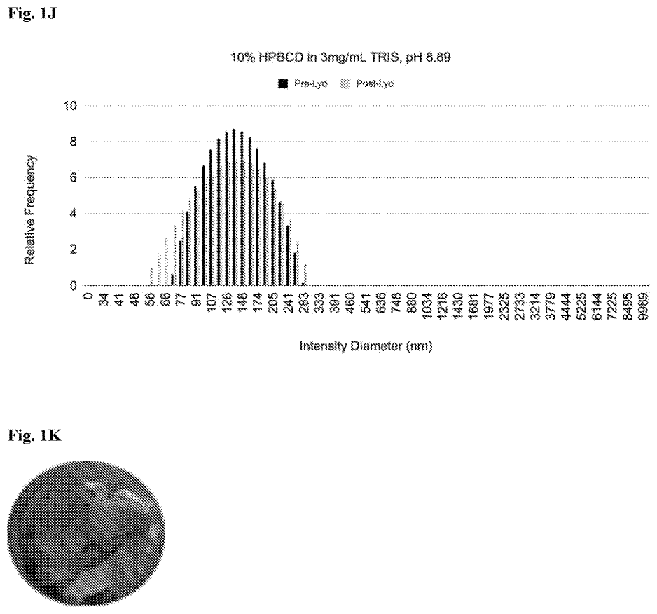

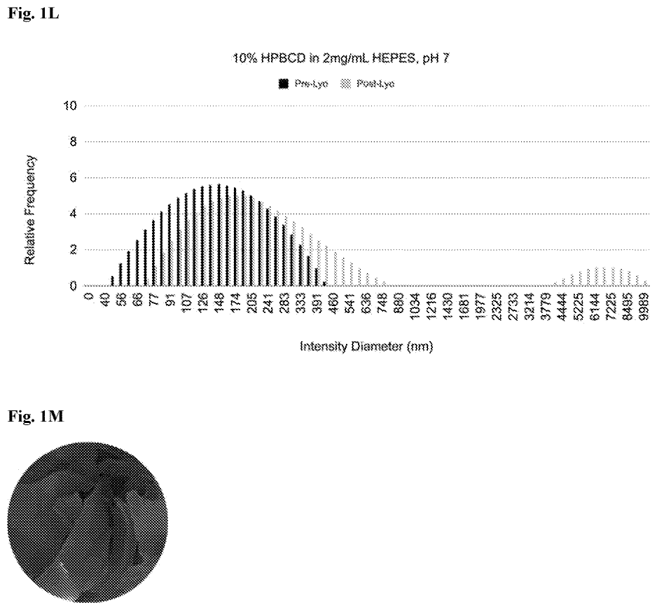

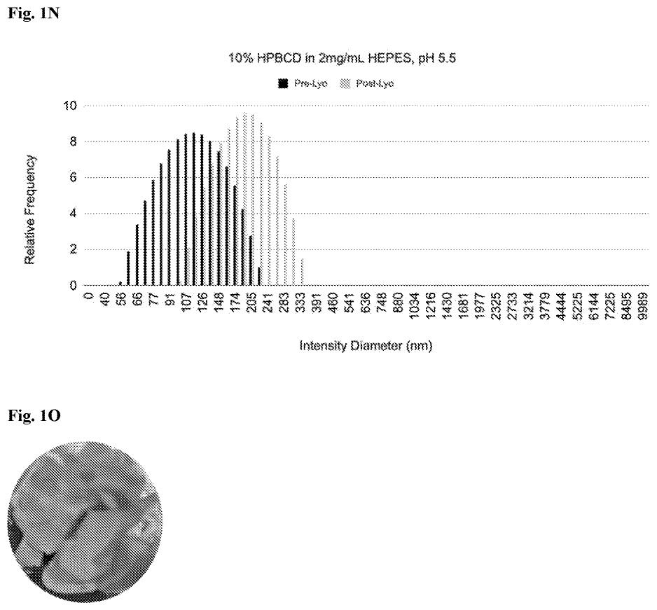

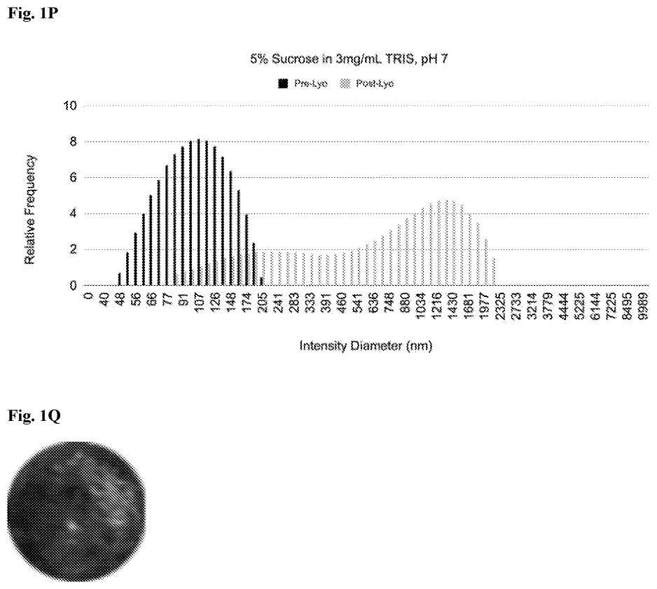

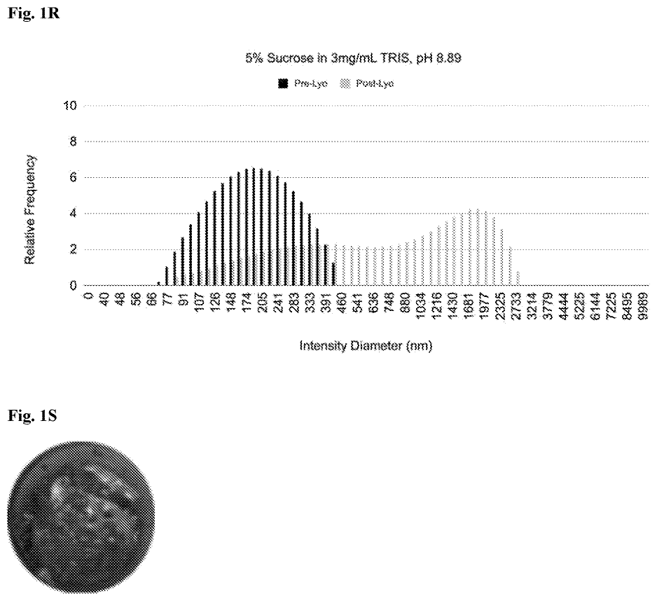

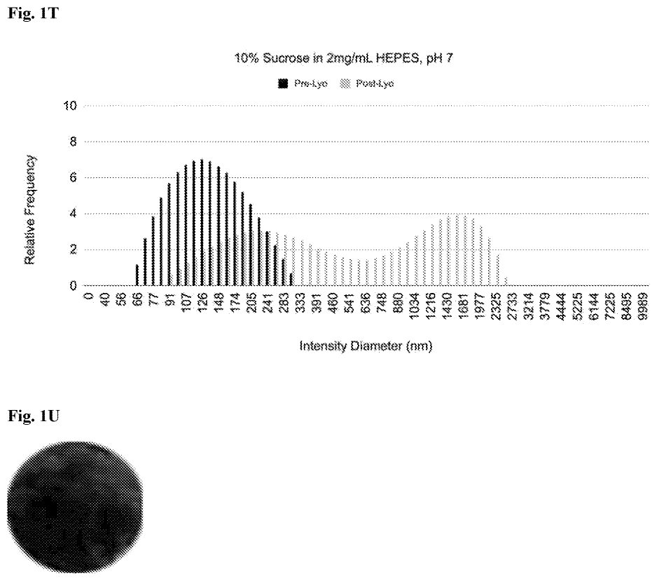

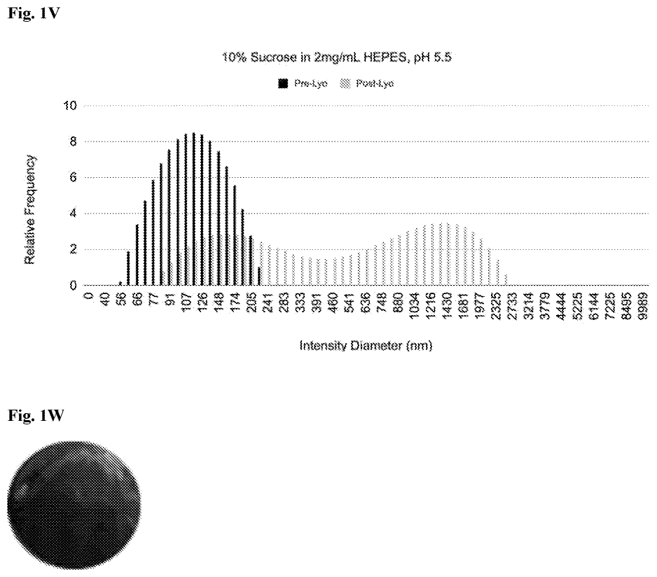

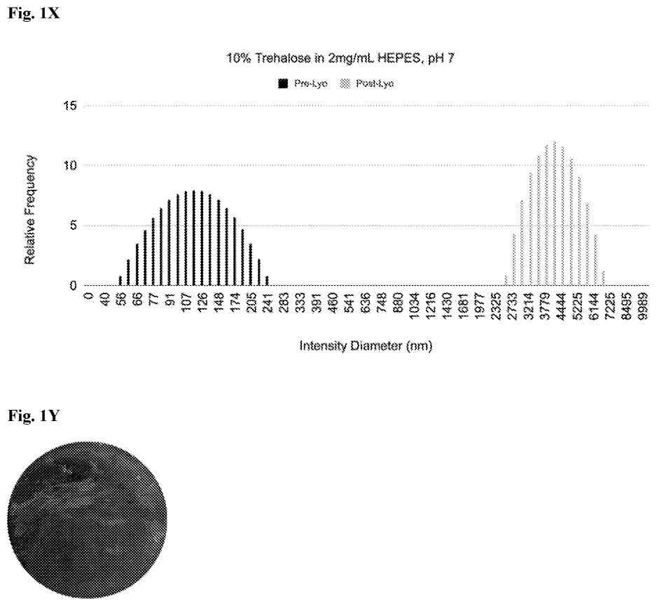

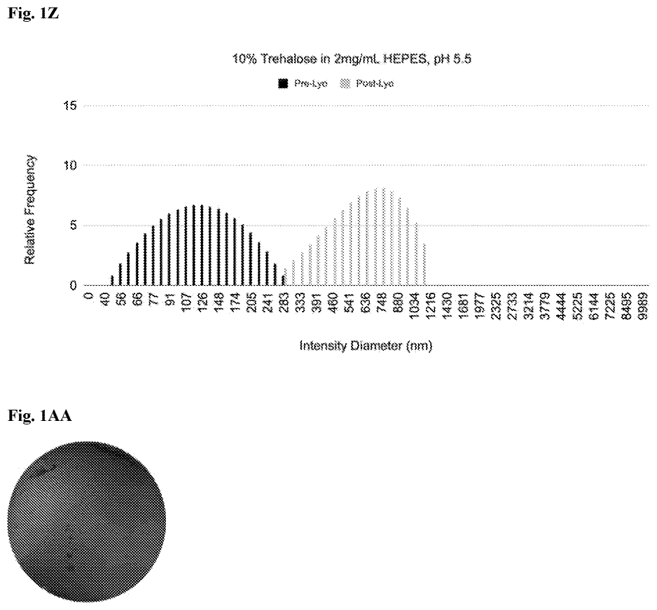

### DETAILED DESCRIPTION

In certain embodiments, the disclosure relates to the use of certain buffers to effectively stabilize during lyophilization a lipid particle (e.g., a liposome) having a polypeptide associated with its outer surface and maintain the functionality and size after reconstitution of the lyophilized lipid particle. In particular, buffers are described herein that comprise a cyclodextrin. Accordingly, compositions comprising a lipid particle with its associated polypeptide(s) and a buffer comprising a cyclodextrin are provided. Methods of lyophilizing such compositions are also provided.

### I. Definitions

The articles “a” and “an” are used herein to refer to one or to more than one (i.e. to at least one) of the grammatical object of the article. By way of example, “an element” means one element or more than one element.

The term “administering” is intended to include routes of administration which allow an agent (such as the compositions described herein) to perform its intended function. Examples of routes of administration for treatment of a body which can be used include injection (subcutaneous, intravenous, parenterally, intraperitoneally, intrathecal, etc.), oral, inhalation, and transdermal routes. The injection can be bolus injections or can be continuous infusion.

Depending on the route of administration, the agent can be coated with or disposed in a selected material to protect it from natural conditions that may detrimentally affect its ability to perform its intended function. The agent may be administered alone, or in conjunction with a pharmaceutically acceptable carrier. The agent also may be administered as a prodrug, which is converted to its active form in vivo. In some embodiments, the agent is orally administered. In other embodiments, the agent is administered through anal and/or colorectal route.

“About” and “approximately” shall generally mean an acceptable degree of error for the quantity measured given the nature or precision of the measurements. Typically, exemplary degrees of error are within 20%, preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

A “conservative substitution” is the substitution of an amino acid with another amino acid with similar physical and chemical properties. In contrast, a “nonconservative substitution” is the substitution of an amino acid with another amino acid with dissimilar physical and chemical properties.

The term “comprise” or variations such as “comprises” or “comprising” will be understood to imply the inclusion of a stated integer (or components) or group of integers (or components), but not the exclusion of any other integer (or components) or group of integers (or components).

As used herein, “homology” is used synonymously with “identity.”

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “hydroxypropyl-beta-cyclodextrin” or “HP-β-CD” refers to a C₆₃H₁₁₂O₄₂ molecule having the following structure:

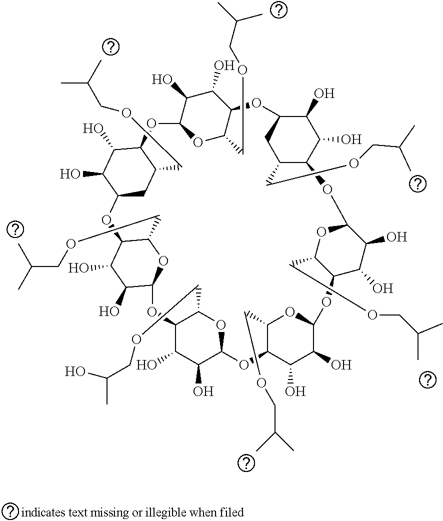 [SMILES: CC(C)COC[C@@H]1C[C@H]2O[C@@H]3[C@@H](O)[C@H](O)[C@H](O[C@@H]4[C@@H](O)[C@H](O)[C@H](O[C@@H]5[C@@H](O)[C@H](O)[C@H](O[C@@H]6[C@@H](O)[C@H](O)[C@H](O[C@H]7[C@H](COCC(C)C)C[C@@H](O[C@@H]8[C@@H](O)[C@H](O)[C@H](O[C@@H]1[C@@H](O)[C@@H]2O)O[C@H]8COCC(C)C)[C@@H](O)[C@@H]7O)O[C@H]6COCC(C)C)O[C@H]5COCC(C)C)O[C@H]4COCC(C)O)O[C@H]3COCC(C)C]

As used herein, “hydroxypropyl-gamma-cyclodextrin” refers to a C₇₂H₁₂₈O₄₈ molecule having the following structure:

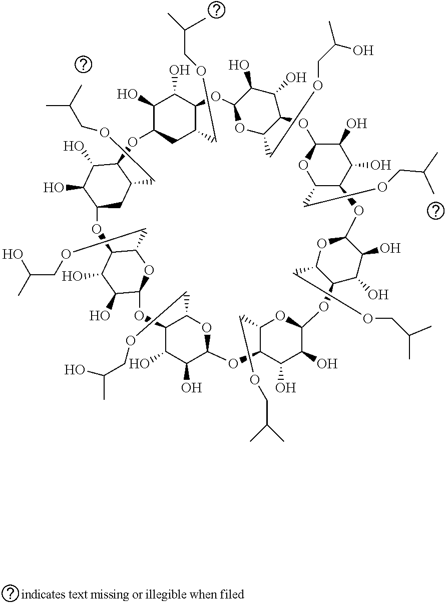 [SMILES: CC(C)COC[C@@H]1C[C@H]2O[C@@H]3[C@@H](O)[C@H](O)[C@H](O[C@@H]4[C@@H](O)[C@H](O)[C@H](O[C@@H]5[C@@H](O)[C@H](O)[C@H](O[C@@H]6[C@@H](O)[C@H](O)[C@H](O[C@@H]7[C@@H](O)[C@H](O)[C@H](O[C@@H]8[C@@H](O)[C@H](O)[C@H](O[C@H]9[C@H](COCC(C)C)C[C@@H](O[C@@H]1[C@@H](O)[C@@H]2O)[C@@H](O)[C@@H]9O)O[C@H]8COCC(C)O)O[C@H]7COCC(C)C)O[C@H]6COCC(C)C)O[C@H]5COCC(C)C)O[C@H]4COCC(C)O)O[C@H]3COCC(C)O]

As used herein, “hydroxypropyl cyclodextrin” refers to a C₃₉H₆₆O₃₁ molecule having the following structure:

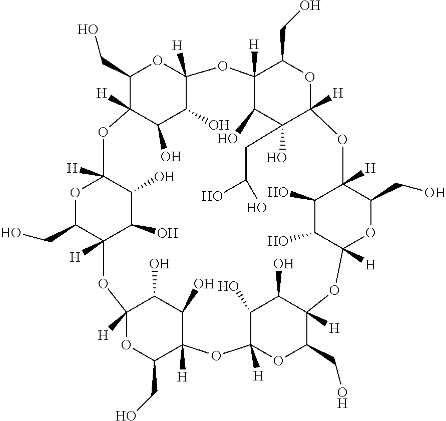 [SMILES: OC[C@H]1O[C@@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]1[C@H](O)[C@]2(O)CC(O)O)O[C@@H]7CO)O[C@@H]6CO)O[C@@H]5CO)O[C@@H]4CO)O[C@@H]3CO]

A “kit” is any manufacture (e.g., a package or container) comprising at least one reagent, e.g. a probe or small molecule, for specifically detecting and/or affecting the expression of a marker. The kit may be promoted, distributed, or sold as a unit for performing the methods described herein. In certain embodiments, the kit may further comprise a reference standard. One skilled in the art can envision many such controls, including, but not limited to, common molecules. Reagents in the kit may be provided in individual containers or as mixtures of two or more reagents in a single container. In addition, instructional materials that describe the use of the compositions within the kit can be included.

“Mutants,” “derivatives,” and “variants” of a polypeptide (or of the DNA encoding the same) are polypeptides which may be modified or altered in one or more amino acids (or in one or more nucleotides) such that the peptide (or the nucleic acid) is not identical to the wild-type sequence, but has homology to the wild type polypeptide (or the nucleic acid).

A “mutation” of a polypeptide (or of the DNA encoding the same) is a modification or alteration of one or more amino acids (or in one or more nucleotides) such that the peptide (or nucleic acid) is not identical to the sequences recited herein, but has homology to the wild type polypeptide (or the nucleic acid).

“Particle” as used herein is meant to include particles, spheres, capsules, and other structures having a length or diameter of about 10 nm to about 10 m. For the purposes of this application, the terms “nanosphere,” “nanoparticle,” “nanocapsule,” “microsphere,” “microparticle,” “microcapsule,” and “particle” are used interchangeably.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

A “portion” of a polypeptide means at least about three sequential amino acid residues of the polypeptide. It is understood that a portion of a polypeptide may include every amino acid residue of the polypeptide.

As used herein, a therapeutic that “prevents” a condition refers to a composition that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

A “recombinant polypeptide” is one, which is produced upon expression of a recombinant polynucleotide.

The term “site” refers to a breach of a surface, for example, the site of an injury, wherein the breach results in von Willebrand Factor and collagen being present at the site.

The term “synergistic effect” refers to the combined effect of two or more agents described herein that is greater than the sum of the separate effects of any one of agents alone.

The term “shelf-life” refers to the time period wherein the lyophilized synthetic platelet composition is able to retain its physicochemical and biofunctional properties after reconstitution within, for example +/−25% compared to pre-lyophilization. These biofunctional properties include, for example, the ability to bind to platelets as shown, for example, by flow cytometry and the ability to provide a hemostatic effect in vivo as shown, for example, by a thrombocytopenic mouse model.

The terms “subject” refer to either a human or a non-human animal. This term includes mammals such as humans, primates, livestock animals (e.g., bovines, porcines), companion animals (e.g., canines, felines) and rodents (e.g., mice, rabbits and rats).

“Treating” a disease in a subject or “treating” a subject having a disease refers to subjecting the subject to a pharmaceutical treatment, e.g., the administration of a drug, such that at least one symptom of the disease is decreased or prevented from worsening.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

Unless otherwise defined herein, scientific and technical terms used in this application shall have the meanings that are commonly understood by those of ordinary skill in the art. Generally, nomenclature and techniques relating to chemistry, molecular biology, cell and cancer biology, immunology, microbiology, pharmacology, and protein and nucleic acid chemistry, described herein, are those well-known and commonly used in the art.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual and partial numbers within that range, for example, 1, 2, 3, 4, 5, 5.5 and 6. This applies regardless of the breadth of the range.

### Lyophilization Buffers

In some aspects of the present invention, compositions are provided that comprise a lipid particle and a liquid component. The liquid component may comprise certain molecules, compounds, compositions, etc., that are useful in buffering the lyophilization of the lipid component. As used herein, the term “lyophilization buffer” refers to a composition that can be added to, combined with, or otherwise associated with a particle, molecule, or composition to be lyophilized (e.g., a lipid particle comprising a polypeptide associated with the external surface of the lipid particle). The buffer may stabilize the composition to be lyophilized such that when reconstituted the composition retains some, most, or all of its biological activity. In some embodiments, the buffer may extend the shelf-life of the lyophilized composition.

In some aspects, compositions comprising a plurality of lipid particles comprising a plurality of polypeptides associated with the external surface of the lipid particle and a liquid component are provided. The liquid component may comprise those elements that buffer the plurality of lipid particles during lyophilization. In some embodiments, the liquid component comprises water and a cyclodextrin. The cyclodextrin can be at a concentration from about 1% (w/v) to about 20% (w/v) of the liquid component. In some embodiments, the cyclodextrin is a hydroxypropyl cyclodextrin. For example, in some embodiments, the cyclodextrin is a hydroxypropyl cyclodextrin (e.g., hydroxypropyl-α-cyclodextrin, hydroxypropyl-β-cyclodextrin (HP-3-CD), and hydroxypropyl-γ-cyclodextrin). In some embodiments, the liquid component comprises more than one hydroxypropyl cyclodextrin.

In some embodiments, the composition comprises an additional agent. For example, the composition described herein can further comprise 4-(2-hydroxyethyl)-1-piperazine ethane sulfonic acid (HEPES), a zwitterionic buffering agent. The concentration of HEPES is between about 0.1% (w/v) to about 0.5% (w/v), about 0.1% (w/v) to about 0.4% (w/v), about 0.1% (w/v) to about 0.3% (w/v), about 0.1% (w/v) to about 0.2% (w/v), about 0.2% to about 0.5% (w/v), about 0.3% (w/v) to about 0.5% (w/v), about 0.4% (w/v) to about 0.5% (w/v) of the lipid component. In some embodiments, the concentration of HEPES 0.1% (w/v) to about 0.5% (w/v) of the liquid component. The pH of the liquid component is, in some embodiment from about 4 to about 10, from about 5 to about 10, from about 6 to about 10, from about 7 to about 10, from about 8 to about 10, from about 9 to about 10, from about 5 to about 9, from about 5 to about 8, from about 5 to about 7, from about 5 to about 6.

In some embodiments, the composition further comprises tris(hydroxymethyl)aminomethane (tris). The tris(hydroxymethyl)aminomethane (tris) can be at a concentration between about 0.1% (w/v) to about 0.5% (w/v), about 0.1% (w/v) to about 0.4% (w/v), about 0.1% (w/v) to about 0.3% (w/v), about 0.1% (w/v) to about 0.2% (w/v), about 0.2% to about 0.5% (w/v), about 0.3% (w/v) to about 0.5% (w/v), about 0.4% (w/v) to about 0.5% (w/v) of the lipid component. In some embodiments, the concentration of tris(hydroxymethyl)aminomethane (tris) 0.1% (w/v) to about 0.5% (w/v) of the liquid component.

In some embodiments, the composition comprises sucrose at a concentration from about 1% (w/v) to about 20% (w/v) (e.g., about 1%, 2%, 3%, 4%, 5%, 6%, 7%, 8%, 9%, 10%, 11%, 12%, 13%, 14%, 15%, 16%, 17%, 18%, 19%, or 20% (w/v)) of the liquid component.

In some embodiments, the composition comprises dextrose at a concentration from about 1% (w/v) to about 20% (w/v) (e.g., about 1%, 2%, 3%, 4%, 5%, 6%, 7%, 8%, 9%, 10%, 11%, 12%, 13%, 14%, 15%, 16%, 17%, 18%, 19%, or 20% (w/v)) of the liquid component.

In some embodiments, the composition comprises sodium chloride at a concentration between about 0.1% (w/v) to about 1.0% (w/v), about 0.1% (w/v) to about 0.9% (w/v), about 0.1% (w/v) to about 0.8% (w/v), about 0.1% (w/v) to about 0.7% (w/v), about 0.1% (w/v) to about 0.6% (w/v), about 0.1% (w/v) to about 0.5% (w/v), about 0.1% (w/v) to about 0.4% (w/v), about 0.1% (w/v) to about 0.3% (w/v), about 0.2% (w/v) to about 1.0% (w/v), about 0.3% (w/v) to about 1.0% (w/v), about 0.4% (w/v) to about 1.0% (w/v), about 0.5% (w/v) to about 1.0% (w/v), about 0.6% (w/v) to about 1.0% (w/v), about 0.7% (w/v) to about 1.0% (w/v), or about 0.8% (w/v) to about 1.0% (w/v) of the liquid component.

In some embodiments, the composition comprises ammonium sulfate at a concentration from about 0.1% (w/v) to about 0.5% (w/v), about 0.1% (w/v) to about 0.4% (w/v), about 0.1% (w/v) to about 0.3% (w/v), about 0.1% (w/v) to about 0.2% (w/v), about 0.2% to about 0.5% (w/v), about 0.3% (w/v) to about 0.5% (w/v), about 0.4% (w/v) to about 0.5% (w/v) of the liquid component.

In some embodiments, the composition comprises ammonium sulfate at a concentration of about 0.1% (w/v) to about 0.5% (w/v) of the liquid component.

In some embodiments, the composition comprises L histidine at a concentration from about 0.1% (w/v) to about 0.5% (w/v), about 0.1% (w/v) to about 0.4% (w/v), about 0.1% (w/v) to about 0.3% (w/v), about 0.1% (w/v) to about 0.2% (w/v), about 0.2% to about 0.5% (w/v), about 0.3% (w/v) to about 0.5% (w/v), about 0.4% (w/v) to about 0.5% (w/v) of the liquid component. In some embodiments, the L histidine is at a concentration from about 0.1% (w/v) to about 0.5% (w/v) of the liquid component.

In some embodiments, the composition comprises lactose monohydrate at a concentration from about 0.1% (w/v) to about 0.5% (w/v), about 0.1% (w/v) to about 0.4% (w/v), about 0.1% (w/v) to about 0.3% (w/v), about 0.1% (w/v) to about 0.2% (w/v), about 0.2% to about 0.5% (w/v), about 0.3% (w/v) to about 0.5% (w/v), about 0.4% (w/v) to about 0.5% (w/v) of the liquid component. In some embodiments, the lactose monohydrate is at a concentration from about 0.1% (w/v) to about 0.5% (w/v) of the liquid component.

In some embodiments, the composition does not comprise at least one of HEPES, tris(hydroxymethyl)aminomethane (tris), sucrose, dextrose, sodium chloride, ammonium sulfate, L-histidine, and lactose monohydrate.

In some embodiments, the dry lyophilized powder component comprises 49% (w/w) to 98% (w/w) of the lyoprotectant cyclodextrin and its derivatives including, but not limited to, hydroxypropyl-β-cyclodextrin (HP-3-CD) and 1% (w/w) to 4% (w/w) 4-(2-hydroxyethyl)-1-piperazine ethanesulfonic acid (HEPES) buffering agent at a pH range of 5 to 9. In certain embodiments, the lyophilization buffers described herein maintain the diameter size range between 30 nm to 300 nm of the lipid particles (e.g., liposomes) after lyophilization. In certain embodiments, these lyophilization buffers also maintain the at least some of the functionality of the decorated binding peptides to bind to activated platelets via Flow Cytometry. The use of HP-β-CD and its corresponding maintenance of size range after lyophilization of liposomes is in contrast to the alternative lyoprotectants of sucrose, dextrose, and trehalose that result in increases in the size range of lyophilized peptide-decorated liposomes, for example. In some embodiments, tris(hydroxymethyl)aminomethane (tris) between 1% (w/w) to 4% (w/w) may be used.

### Lipid Particles

The compositions disclosed herein comprise lipid particles comprising a lipid and a peptide conjugate, wherein the peptide of the peptide conjugate is associated with the external surface of the lipid particle. In some embodiments, the lipid in the peptide conjugate is DSPE-PEG(2 k)-maleimide.

The lipid particles in the composition may vary in size. For example, the lipid particles can have a diameter from about 1 nm to about 1000 nm, about 1 nm to about 900 nm, about 1 nm to about 800 nm, about 1 nm to about 700 nm, about 1 nm to about 600 nm, about 1 nm to about 500 nm, about 1 nm to about 400 nm, about 1 nm to about 300 nm, about 1 nm to about 200 nm, about 1 nm to about 100 nm, about 1 nm to about 50 nm, about 1 nm to about 25 nm, about 25 nm to about 1000 nm, about 50 nm to about 1000 nm, about 100 nm to about 1000 nm, about 200 nm to about 1000 nm, about 300 nm to about 1000 nm, about 400 nm to about 1000 nm, about 500 nm to about 1000 nm, about 600 nm to about 1000 nm, about 700 nm to about 1000 nm, about 800 nm to about 1000 nm, or about 900 nm to about 1000 nm. In some embodiments, the lipid particles have an average diameter from about 10 nm to about 500 nm, about 50 nm to about 500 nm, about 100 nm to about 500 nm, about 200 nm to about 500 nm, about 300 nm to about 500 nm, about 400 nm to about 500 nm, about 20 nm to about 400 nm, about 20 nm to about 300 nm, about 20 nm to about 200 nm, about 20 nm to about 100 nm, or about 20 nm to about 50 nm. In some embodiments, the lipid particles have an average diameter from about 30 nm to about 300 nm.

In some embodiments, the lipid particles have a polydispersity index from about 0.1 to about 0.5, about 0.2 to about 0.5, about 0.3 to about 0.5, about 0.4 to about 0.5, about 0.1 to about 0.4, about 0.1 to about 0.3, or about 0.1 to about 0.2. In some embodiments, the lipid particles have a polydispersity index no greater than 0.1, 0.2, 0.3, 0.4, or 0.5. In some embodiments, the lipid particles have a polydispersity index no greater than 0.3.

In some embodiments, the lipid particles have a substantially spherical morphology.

In some embodiments, the lipid particles have a net positive or net negative zeta potential.

The lipid particles comprise a peptide associated with the external surface of the lipid particle. In some embodiments, the lipid particles comprise a phospholipid conjugated to polyethylene glycol (PEG). In some embodiments, the PEG conjugated to the phospholipid has an average molecular weight from about 100 Da to about 10,000 Da, about 500 Da to about 10,000 Da, about 1,000 Da to about 10,000 Da, about 2,000 Da to about 10,000 Da, about 3,000 Da to about 10,000 Da, about 4,000 Da to about 10,000 Da, about 5,000 Da to about 10,000 Da, about 6,000 Da to about 10,000 Da, about 7,000 Da to about 10,000 Da, about 8,000 Da to about 10,000 Da, about 9,000 Da to about 10,000 Da, about 100 Da to about 9,000 Da, about 100 Da to about 8,000 Da, about 100 Da to about 7,000 Da, about 100 Da to about 6,000 Da, about 100 Da to about 5,000 Da, about 100 Da to about 4,000 Da, about 100 Da to about 3,000 Da, about 100 Da to about 2,000 Da, or about 100 Da to about 1,000 Da In some embodiments, In some embodiments, the PEG conjugated to the phospholipid has an average molecular weight from about 500 Da to about 5500 Da.

In some embodiments, the phospholipid is distearoylphosphatidylcholine (DSPC) or 1,2-distearoyl-sn-glycero-3-phosphoethanolamine (DSPE). In some embodiments, the lipid particles comprise cholesterol.

In some embodiments, the lipid particles are liposomes.

### Peptides

In some embodiments, the lipid particles (e.g., liposomes) are decorated with peptides. This peptide decoration of the liposomes comprises of phospholipids conjugated to polyethylene glycol (PEG) and conjugated to a binding peptide that is formulated with phospholipid-PEG, phospholipids and optionally lipids. These phospholipids comprise, for example, distearoylphosphatidylcholine (DSPC) and its derivatives and 1,2-distearoyl-sn-glycero-3-phosphoethanolamine (DSPE) and its derivatives. In some embodiments, the lipids comprise cholesterol and its derivatives. In some embodiments, the phospholipid-PEG conjugates comprise PEG with molecular weights between 1000 Da to 2000 Da.

In some embodiments, each lipid particle comprises a plurality of peptides associated with the external surface of the lipid particle. For example, a lipid particle can comprise a first peptide and second peptide. In some embodiments, this lipid particle can also comprise a third peptide. The first peptide may have an affinity for a first target, the second peptide may have an affinity for a second target, and the third peptide, if present, may have an affinity for a third target.

In some embodiments, the polypeptide decorated lipid particles are synthetic platelets.

In certain embodiments, the peptides are
platelet binding proteins (PBPs), for example P-selectin binding peptides (DAEWVDVS (SEQ ID NO: 5)) or fibrinogen mimetic peptides (FMPs), such as FMPs of Formula (I) (also referred to herein as FMP2; cyclo-(CNPRGD{Tyr(OEt)}R-β-A) (SEQ ID NO: 1)), FMP1 (cyclo-{Pra}CNPRGD{Tyr(OEt)}RC (SEQ ID NO: 2)), linear RGD (GRGDSP (SEQ ID NO: 3)), and H12 (HHLGGAKQAGDV (SEQ ID NO: 4));
collagen binding peptides (CBPs), for example (GPO)₇ (SEQ ID NO: 8); or
von Willebrand binding proteins (VBPs), for example TRYLRIHPQSWVHQI (SEQ ID NO: 6),
or combinations thereof, or their conjugates thereof, or combinations of conjugates thereof. In addition, compositions and methods for using PBPs, CBPs, and VBPs and particles conjugated to these peptides are also provided.

### Platelet Binding Proteins (PBPs)

### Fibrinogen Mimetic Peptides (FMPs)

As used herein, the terms “fibrinogen mimetic peptide” and the term “active platelet GPIIb-IIIa-binding peptide” are used interchangeably in the present disclosure. In some aspects, provided herein is a fibrinogen mimetic peptide of Formula (I):
cyclo-(CNPRGD{Tyr(OEt)}R-β-A) (SEQ ID NO: 1), referred to herein as “FMP2”, or a salt thereof.

In some embodiments, the fibrinogen mimetic peptide is FMP1, which has the formula: cyclo-(CNPRGD{Tyr(OEt)}R-β-C) (SEQ ID NO: 2), or a salt thereof.

In some embodiments, the FMP is linear RGD (GRGDSP (SEQ ID NO: 3)).

In some embodiments, the FMP is H12 (HHLGGAKQAGDV (SEQ ID NO: 4)).

In some embodiments, the salt of the fibrinogen mimetic is an acetate salt, or a trifluoroacetate salt. In some embodiments, the fibrinogen mimetic peptide specifically binds to activated GPIIb-IIIa. In some embodiments, the fibrinogen mimetic peptide inhibits platelet aggregation. In some embodiments, the fibrinogen mimetic peptide has an IC₅₀ less than 30 μM, e.g., less than 25 μM, less than 20 μM, less than 15 μM, less than 10 μM, less than 5 μM, less than 1 μM, less than 0.5 μM, less than 0.1 μM, less than 0.05 μM, less than 4.5×10⁻² M, less than 4.0×10⁻² M, less than 3.5×10⁻² M, less than 3.0×10⁻² μM, less than 2.5×10⁻² M, less than 2.0×10⁻² M, less than 1.5×10⁻² M, less than 1.0×10⁻² μM, less than 0.5×10⁻² M, less than 0.1×10⁻² μM, less than 0.5×10⁻³ μM, less than 0.1×10⁻³ μM, etc. In certain embodiments, the fibrinogen mimetic peptide has an IC₅₀ of about 0.13 μM.

In some embodiments, the fibrinogen mimetic peptide disclosed herein is conjugated to a polymer (e.g., a lipid, a protein, etc.). Therefore, in some embodiments, provided herein are fibrinogen mimetic peptide conjugates comprising a fibrinogen mimetic peptide of SEQ ID NO: 1, 2, 3, or 4 conjugated to a polymer. In some embodiments, the polymer is a lipid (e.g., DSPE-PEG(2 k)-maleimide). In some embodiments, the fibrinogen mimetic peptide is conjugated to the polymer through thio-ene coupling to the thiol group on an existing or added N-terminal cysteine or 3-mercaptopropionic acid. In some embodiments, the surface of a particle (e.g., a synthetic platelet) comprises an FMP peptide, or a salt thereof.

In some embodiments, the FIMP can include an RGD amino acid sequence motif that promotes active platelet aggregation. The RGD motif containing FMP may contain a single repeat of the RGD motif or may contain multiple repeats of the RGD motif, such as, for example, 2, 3, 4, 5, 6, 7, 8, 9, 10, or more repeats of the RGD motif. One of skill in the art will understand that conservative substitutions of particular amino acid residues of the RGD motif containing FMPs may be used so long as the RGD motif containing FIMP retains the ability to bind comparably as the native RGD motif. One of skill in the art will also understand that conservative substitutions of particular amino acid residues flanking the RGD motif so long as the RGD motif containing FIMP retains the ability to bind comparably to the native RGD motif.

In some embodiments, the FIMP can be a fibrinogen mimetic peptide (FMP) described herein. In some embodiments, FMP is of Formula (I). A cyclic peptide of Formula (I) can have high selectivity and affinity to GPIIb-IIIa on activated platelets but do not bind or activate quiescent platelets nor interact with other RGD-binding integrins. The FMP can be synthesized using Fmoc-based solid phase chemistry on Knorr resin and characterized using mass spectroscopy.

Advantageously, the FMPs can each include about 5 to about 30 amino acids. By limiting the size of the peptides to about 5 to about 30 amino acids, the FMPs can be spatially or topographically arranged on the flexible particle surface such that the FMPs do not spatially mask each other and are able to promote arrest and aggregation of active platelets onto injury sites.

### P-Selectin Binding Peptide

As used herein, the term “P-selectin binding peptide” refers to a protein or peptide that binds to P-selectin on platelets with high affinity (e.g., nano-micromolar affinity), such as glycosulfopeptide mimics of the N-terminal of PSGL-1 (native ligand for P-selectin) or peptides derived from phage display such as EWVDV-containing peptides. In certain embodiments, the P-selectin binding peptide has the amino acid sequence DAEWVDVS (SEQ ID NO: 5).

In some embodiments, the P-selectin binding peptide disclosed herein is conjugated to a polymer (e.g., a lipid, a protein, etc.). Therefore, in some embodiments, provided herein are P-selectin binding peptide conjugates comprising a P-selectin binding peptide of SEQ ID NO: 5 conjugated to a polymer. In some embodiments, the polymer is a lipid (e.g., DSPE-PEG(2 k)-maleimide). In some embodiments, the P-selectin binding peptide is conjugated to the polymer through thio-ene coupling to the thiol group on an added N-terminal cysteine or 3-mercaptopropionic acid. In some embodiments, the surface of a particle (e.g., a synthetic platelet) comprises a P-selectin binding peptide, or a salt thereof.

In some embodiments, the P-selectin binding peptide can be a peptide described herein. In specific embodiments, the P-selectin binding peptide having the amino acid sequence of DAEWVDVS (SEQ ID NO: 5). A peptide having the amino acid sequence of SEQ ID NO: 5 can have high selectivity and affinity to P-selectin on activated platelets. The P-selectin binding peptide can be synthesized using FMoc-based solid phase chemistry on Knorr resin and characterized using mass spectroscopy.

Advantageously, the P-selectin binding peptide can each include about 5 to about 30 amino acids. By limiting the size of the peptides to about 5 to about 30 amino acids, the P-selectin binding peptides can be spatially or topographically arranged on the flexible particle surface such that they do not spatially mask each other and are able to promote arrest and aggregation of active platelets onto injury sites.

### von Willebrand Binding Peptide (VBP)

As used herein, the term “von Willebrand binding peptide” refers to a protein or peptide that binds to von Willebrand Factor with high affinity (e.g., nano-micromolar affinity). Von Willebrand Factor has multiple binding domains, so the VBP could consist of a peptide that binds to the D′D3 domain (such as Factor FVIII-derived peptides), the A1 or A3 domains (such as collagen-derived/mimetic peptides), or A1 or C4 domains (such as platelet GPIb or GPIIb-IIIa-derived peptides). In certain embodiments, the VBP has the amino acid sequence TRYLRIHPQSWVHQI (SEQ ID NO: 6).

In some embodiments, the VBP disclosed herein is conjugated to a polymer (e.g., a lipid, a protein, etc.). Therefore, in some embodiments, provided herein are VBP conjugates comprising a VBP of SEQ ID NO: 6 conjugated to a polymer. In some embodiments, the polymer is a lipid (e.g., DSPE-PEG(2 k)-maleimide). In some embodiments, the VBP is conjugated to the polymer through thio-ene coupling to the thiol group on an added N-terminal cysteine or 3-mercaptopropionic acid. In some embodiments, the surface of a particle (e.g., a synthetic platelet) comprises a VBP, or a salt thereof.

In some embodiments, the VBP peptide for vWF binding can include a recombinant GPIba fragment (rGPIba) containing the vWF binding sites (residues 1 to 302) or a short chain vWF-binding peptide. The GPIba fragment can be expressed in CHO cells and isolated, adapting methods described. The short VBP can comprise the amino acid sequence TRYLRIHPQSWVHQI (SEQ ID NO: 6), A peptide having an amino acid sequence of SEQ ID NO: 6 can be synthesized using fluorenylmethyloxycarbonyl chloride (FMoc)-based solid phase chemistry on Knorr resin, and characterized using mass spectroscopy. Each vWF molecule has only one binding region for this peptide, and hence vascular injury sites presenting multiple vWF binding sites for multiple copies of this peptide decorated on the particle surface provide a mechanism for enhanced adhesion of the particles with increasing shear.

### Collagen Binding Peptide (CBP)

As used herein, the term “collagen_binding peptide” refers to a protein or peptide that binds to collagen with high affinity (e.g., nano-micromolar affinity), such as collagen-derived sequences (such as GPO repeats) that have hellicogenic affinity for collagen or collagen binding peptides derived experimentally (such as phage display or isothermal titration chemistry). In certain embodiments, the CBP has the amino acid sequence (GPO)₇ (SEQ ID NO: 7).

In some embodiments, the CBP disclosed herein is conjugated to a polymer (e.g., a lipid, a protein, etc.). Therefore, in some embodiments, provided herein are CBP conjugates comprising a CBP of SEQ ID NO: 7 conjugated to a polymer. In some embodiments, the polymer is a lipid (e.g., DSPE-PEG(2 k)-maleimide). In some embodiments, the CBP is conjugated to the polymer through thio-ene coupling to the thiol group on an added N-terminal cysteine or 3-mercaptopropionic acid. In some embodiments, the surface of a particle (e.g., a synthetic platelet) comprises a CBP, or a salt thereof.

In some embodiments, the CBP can comprise a peptide that comprises a short repeat of the tripeptide GPO, such as (GPO)₇ SEQ ID NO: 7, with a helicogenic affinity to fibrillar collagen. The GPO trimer is based on amino acid repeats found in the native collagen structure. It has been reported that the activation of platelets usually caused by interaction with collagen through GPVI and GPIa/IIa, can also potentially occur when platelets interact with collagen-derived peptides. This can be a potential problem regarding decorating synthetic particle surfaces with collagen-derived peptides for binding of collagen because in vivo the constructs can potentially interact with quiescent blood platelets and systemically activate them, posing thromboembolic risks. However, interaction of platelet receptors with collagen and the subsequent platelet activation mechanisms are dependent upon receptor clustering induced by multimeric long chain triple-helical fibrillar collagen and not by short collagen-mimetic peptide repeats. In fact, it has been shown that GPO-trimer repeats as high as a 30-mer (10 repeats) only partially interact with platelet GPIa/IIa and GPVI integrins and are incapable of activating platelets; yet they can effectively bind to fibrillar collagen via helicogenic interaction. Hence, this small CBP can promote adhesion to fibrillar collagen, but cannot activate quiescent platelets due to absence of long triple-helical conformation. The CBP like the VBP can also be synthesized using FMoc-based solid phase chemistry on Knorr resin and characterized using mass spectroscopy.

### Synthetic Platelets and Uses

In some aspects, the present disclosure relates to lipid particles that function as synthetic platelets. In certain embodiments, the particles are conjugated to a plurality of Platelet Binding Peptides (PBPs), such as FMP1 peptides, FMP2 peptides, linear RGD peptides, H12 peptides, and/or P-selectin binding peptides; CBPs; and VBPs described herein. For example, in some embodiments, the particle is conjugated to an FMP1 peptide. In some embodiments, the particle is conjugated to an FMP2 peptide. In some embodiments, the particle is conjugated to a linear RGD peptide. In some embodiments, the particle is conjugated to an H12 peptide. In some embodiments, the particle is conjugated to a P-selectin binding peptide. In some embodiments, the particle is conjugated to a CBP. In some embodiments, the particle is conjugated to a VBP. Methods of using these particles in diminishing bleeding and blood loss are provided, as well as compositions and methods useful in the delivery of therapeutic agents to the vasculature. The synthetic platelets described herein integrate platelet-mimetic adhesion- and aggregation-promoting functionalities on a single flexible particle. It was found that the platelet-mimetic adhesion- and aggregation-promoting functionalities can be achieved by including on, conjugating to, or decorating a flexible particle with a plurality of three peptides, i.e., a VBP, a CBP, and a PBP. It was initially found that liposomes bearing VBP and CBP motifs undergo platelet-mimetic adhesion under flow on vWF and collagen-coated surfaces in vitro at low-to-high shear in parallel plate flow chamber (PPFC) experiments and that PBP-modified liposomes pre-adhered to a surface can enhance the aggregation of ADP-activated platelets onto them, even at low platelet concentrations. Subsequently, it was found that liposomes bearing all three peptides (VBP, CBP and PBP), when introduced in PPFC flow along with low concentration of ADP-activated platelets over a vWF/collagen mixed coated surface, are able to adhere to the surface under high shear and promote arrest and aggregation of active platelets onto sites of liposome adhesion. In some embodiments, the PBP included in the synthetic platelets is a fibrinogen mimetic peptide (FMP) described herein. In some embodiments, the FMP is FMP1, FMP2, linear RGD, or H12, or a combination thereof. In some embodiments, the PBP included in the synthetic platelets is a P-selectin binding peptide described herein.

It is therefore an aspect of the application that administration, such as intravenous administration, of the synthetic platelets described herein to a subject with a vascular injury can diminish the bleeding time in the subject. It is a further aspect of the application that the synthetic platelets provide a nanostructure that binds with a vascular injury site as well as activated platelets and enhances their rate of adhesion and aggregation to aid in stopping bleeding.

In some embodiments, the synthetic platelets described herein can include a biocompatible, biodegradable, flexible particle core and a plurality of VBPs, CBPs, and PBPs bound to, conjugated to, and/or decorated on the a surface defined by the flexible particle core. The VBPs, CBPs, and PBPs can be spatially or topographically arranged on the flexible particle surface such that the VBPs, CBPs, and PBPs do not spatially mask each other and are able to adhere to a vascular surface, vascular disease site, and/or vascular injury site with exposed vWF and collagen and promote arrest and aggregation of active platelets onto sites of particle adhesion.

The biocompatible, biodegradable, flexible particles be made from any biocompatible, biodegradable material that can form a flexible particle to which the peptides described herein can be attached, conjugated, and/or decorated. In some embodiments, the biocompatible, biodegradable flexible particles can include a liposome, a hydrogel, micelle, and/or polymer, which can include and/or be surface modified or engineered with the VBPs, CBPs, and PBPs.

The liposome or hydrogel can include a lipid and/or any naturally occurring, synthetic or semi-synthetic (i.e., modified natural) moiety that is generally amphipathic (i.e., including a hydrophilic component and a hydrophobic component). Examples of lipids can include fatty acids, neutral fats, phospholipids, oils, glycolipids, surfactants, aliphatic alcohols, waxes, terpenes and steroids. Semi-synthetic or modified natural lipids can include natural lipids that have been chemically modified in some fashion. The at least one lipid can be neutral, negatively charged (i.e., anionic), or positively charged (i.e., cationic). Examples of anionic lipids can include phosphatidic acid, phosphatidyl glycerol, and fatty acid esters thereof, amides of phosphatidyl ethanolamine, such as anandamides and methanandamides, phosphatidyl serine, phosphatidyl inositol and fatty acid esters thereof, cardiolipin, phosphatidyl ethylene glycol, acidic lysolipids, sulfolipids and sulfatides, free fatty acids, both saturated and unsaturated, and negatively charged derivatives thereof. Examples of cationic lipids can include N-[1-(2,3-dioleoyloxy)propyl]-N,N,N-trimethyl-ammonium chloride and common natural lipids derivatized to contain one or more basic functional groups.

Other examples of lipids, any one or combination of which may be used to form the particle, can include: phosphocholines, such as 1-alkyl-2-acetoyl-sn-glycero 3-phosphocholines, and 1-alkyl-2-hydroxy-sn-glycero 3-phosphocholines; phosphatidylcholine with both saturated and unsaturated lipids, including dioleoylphosphatidylcholine, dimyristoylphosphatidylcholine, dipentadecanoylphosphatidylcholine, dilauroylphosphatidylcholine, dipalmitoylphosphatidylcholine (DPPC), distearoylphosphatidylcholine (DSPC), and diarachidonylphosphatidylcholine (DAPC); phosphatidylethanolamines, such as dioleoylphosphatidylethanolamine, dipalmitoylphosphatidylethanolamine (DPPE), and distearoylphosphatidylethanolamine (DSPE); phosphatidylserine; phosphatidylglycerols, including distearoylphosphatidylglycerol (DSPG); phosphatidylinositol; sphingolipids, such as sphingomyelin; glycolipids, such as ganglioside GM1 and GM2; glucolipids; sulfatides; glycosphingolipids; phosphatidic acids, such as dipalmitoylphosphatidic acid (DPPA) and distearoylphosphatidic acid (DSPA); palmitic acid; stearic acid; arachidonic acid; oleic acid; lipids bearing polymers, such as chitin, hyaluronic acid, polyvinylpyrrolidone or polyethylene glycol (PEG); lipids bearing sulfonated mono-, di-, oligo- or polysaccharides; cholesterol, cholesterol sulfate, and cholesterol hemisuccinate; tocopherol hemisuccinate; lipids with ether and ester-linked fatty acids; polymerized lipids (a wide variety of which are well known in the art); diacetyl phosphate; dicetyl phosphate; stearylamine; cardiolipin; phospholipids with short chain fatty acids of about 6 to about 8 carbons in length; synthetic phospholipids with asymmetric acyl chains, such as, for example, one acyl chain of about 6 carbons and another acyl chain of about 12 carbons; ceramides; non-ionic liposomes including niosomes, such as polyoxyalkylene (e.g., polyoxyethylene) fatty acid esters, polyoxyalkylene (e.g., polyoxyethylene) fatty alcohols, polyoxyalkylene (e.g., polyoxyethylene) fatty alcohol ethers, polyoxyalkylene (e.g., polyoxyethylene) sorbitan fatty acid esters (such as, for example, the class of compounds referred to as TWEEN (commercially available from ICI Americas, Inc., Wilmington, Del.), glycerol polyethylene glycol oxystearate, glycerol polyethylene glycol ricinoleate, alkyloxylated (e.g., ethoxylated) soybean sterols, alkyloxylated (e.g., ethoxylated) castor oil, polyoxyethylene-polyoxypropylene polymers, and polyoxyalkylene (e.g., polyoxyethylene) fatty acid stearates; sterol aliphatic acid esters including cholesterol sulfate, cholesterol butyrate, cholesterol isobutyrate, cholesterol palmitate, cholesterol stearate, lanosterol acetate, ergosterol palmitate, and phytosterol n-butyrate; sterol esters of sugar acids including cholesterol glucuronide, lanosterol glucuronide, 7-dehydrocholesterol glucuronide, ergosterol glucuronide, cholesterol gluconate, lanosterol gluconate, and ergosterol gluconate; esters of sugar acids and alcohols including lauryl glucuronide, stearoyl glucuronide, myristoyl glucuronide, lauryl gluconate, myristoyl gluconate, and stearoyl gluconate; esters of sugars and aliphatic acids including sucrose laurate, fructose laurate, sucrose palmitate, sucrose stearate, glucuronic acid, gluconic acid and polyuronic acid; saponins including sarsasapogenin, smilagenin, hederagenin, oleanolic acid, and digitoxigenin; glycerol dilaurate, glycerol trilaurate, glycerol dipalmitate, glycerol and glycerol esters including glycerol tripalmitate, glycerol distearate, glycerol tristearate, glycerol dimyristate, glycerol trimyristate; long chain alcohols including n-decyl alcohol, lauryl alcohol, myristyl alcohol, cetyl alcohol, and n-octadecyl alcohol; 6-(5-cholesten-3-yloxy)-1-thio-D-galactopyranoside; digalactosyldiglyceride; 6-(5-cholesten-3-yloxy)hexyl-6-amino-6-deoxy-1-thio-D-galactopyranoside; 6-(5-cholesten-3-yloxy)hexyl-6-amino-6-deoxyl-1-thio-a-D-mannopyranoside; 12-(((7′-diethylaminocoumarin-3-yl)carbonyl)methylamino)octadecanoic acid; N-[12-(((7′-diethylaminocoumarin-3-yl)carbonyl)methylamino)octadecanoyl]-2-aminopalmitic acid; cholesteryl(4′-trimethylammonio)butanoate; N-succinyldioleoylphosphatidylethanolamine; 1,2-dioleoyl-sn-glycerol; 1,2-dipalmitoyl-sn-3-succinylglycerol; 1,3-dipalmitoyl-2-succinylglycerol; 1-hexadecyl-2-palmitoylglycerophosphoethanolamine and palmitoylhomocysteine; and/or any combinations thereof.

Examples of biocompatible, biodegradable polymers that can be used to form the particles are poly(lactide)s, poly(glycolide)s, poly(lactide-co-glycolide)s, poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and poly(lactide)s or poly(lactide-co-glycolide)s, biodegradable polyurethanes, and blends and/or copolymers thereof.

Other examples of materials that may be used to form the particles can include chitosan, poly(ethylene oxide), poly(lactic acid), poly(acrylic acid), poly(vinyl alcohol), poly(urethane), poly(N-isopropyl acrylamide), poly(vinyl pyrrolidone) (PVP), poly(methacrylic acid), poly(p-styrene carboxylic acid), poly(p-styrenesulfonic acid), poly(vinylsulfonicacid), poly(ethyleneimine), poly(vinylamine), poly(anhydride), poly(L-lysine), poly(L-glutamic acid), poly(gamma-glutamic acid), poly(carprolactone), polylactide, poly(ethylene), poly(propylene), poly(glycolide), poly(lactide-co-glycolide), poly(amide), poly(hydroxylacid), poly(sulfone), poly(amine), poly(saccharide), poly(HEMA), poly(anhydride), gelatin, glycosaminoglycans (GAG), poly(hyaluronic acid), poly(sodium alginate), alginate, albumin, hyaluronan, agarose, polyhydroxybutyrate (PHB), copolymers thereof, and blends thereof.

The flexible particles can have a maximum length or diameter of about 100 nm to about m and a substantially spherical, discoidal, and/or ellipsoidal shape. The physical size and shape as well as mechanical properties of the particles can be engineered to mimic those of natural platelets that are important in hemostasis. In some embodiments, the flexible particles can have an about 2 to about 5 μm diameter discoidal shape and an about 10 to about 50 kPa mechanical elastic modulus that mimics the size, shape, and elastic modulus of platelets and facilitates upon administration to the vasculature of a subject their margination to the vascular wall and their bio-interactions.

In an embodiment of the application, oblate ellipsoid particles having a diameter of about 2 to about 5 μm and a mechanical modulus of about 10 to about 50 kPa can be prepared by initially forming a polymer template. The polymer template can then be used to build a protein/polymer shell using a cross-linked layer-by-layer assembly. The polymer template can subsequently be removed using solvents to leave behind soft, flexible, proteinaceous discoid particles having a diameter about 2 to about 5 μm and a mechanical elastic modulus of about 10 to about 50 kPa. The particles can then be surface-modified with the VBPs, CBPs, and PBPs at a surface density effective to promote maximum particle adhesion to vWF and collagen exposed surfaces at low-to-high sheer stresses and promote aggregation of active platelets even at low (less about 50,000 per 1) platelet concentrations.

By way of example, poly-L-lactide-co-glycolide (PLGA) spherical particles having a diameter of about 2 to about 3 μm can be embedded into polyvinyl alcohol (PVA) film (e.g., about 5% w/v in water) containing 2% (v/v) glycerol as a plasticizer and biaxially stretched to twice the original length and width in an oven at about 65° C. The film can be removed from the stretcher and the PVA dissolved in 15% isopropanol followed by thorough washing with isopropanol to ensure complete removal of PVA. This results in the recovery of the oblate PLGA particles that can be resuspended in distilled water or PBS. These template particles can then be coated with protein and polyelectrolyte layers using a layer-by-layer (LBL) technique. For this, protein serum albumin (SA, e.g., human serum albumin or mouse serum albumin) and the polyelectrolyte polyallylamine hydrochloride (PAH) can be used at a 2 mg/ml concentration for adsorption. At the pH employed, albumin is negatively charged and PAH is cationic, alternate layers of SA and PAH can be formed on the PLGA template particles via electrostatic interactions. Multiple alternating layers (e.g., at least seven layers) can be formed on the oblate template and cross-linked with gluteraldehyde intermittently to enhance stability. The particles can then be exposed to a solvent mixture (e.g., 2:1 tetrahydrofuran:isopropanol) to dissolve the PLGA core, leaving behind the LBL deposited soft SA/PAH flexible discoid shell. The outermost layer can include albumin so that PEGylated peptides describe herein can be readily attached.

The VBPs, CBPs, and PBPs can be conjugated to the particle surface by reacting the peptides through a thiol group on an existing or added N-terminal cysteine or 3-mercaptopropionic acid to a maleimide-terminated lipid, such as maleimide-PEG-DSPE. The lipid-peptide conjugates can then be incorporated into lipophilic particles such as liposomes using known formulation techniques.

The VBPs, CBPs, and PBPs can be conjugated to the particle surface by reacting the peptides with through their N-termini to the carboxyl termini of a heterobifunctional PEG, such as maleimide-PEG-COOH. The PEG-peptide conjugates or PEGylated peptides can then be conjugated to the particle using known conjugation techniques.

The PEG molecules can have a variety of lengths and molecular weights, including, for example, PEG 200, PEG 1000, PEG 1500, PEG 2000, PEG 4600, PEG 10,000, or combinations thereof. In other embodiments, the VBPs, CBPs, and PBPs can be conjugated to the particle surface with PEG acrylate, or PEG diacrylate, molecules of a variety of molecular weights.

In one example, the VBPs, CBPs, and PBPs can be reacted with maleimide-PEG-COOH to form Mal-PEG-peptide conjugates. SA/PAH particles with albumin as the outermost layer can then be treated with dithiothreitol (DTT) to introduce a high density of sulfhydryl (—SH) groups on the surface. The Mal-PEG-peptides can then be incubated with the DTT-treated particles, such that the MAL termini can react with the free SH groups to form particles decorated with various peptides presented on the particle surface via PEG linkers.

The relative amounts of the peptides conjugated to the particle surface can affect the efficiency of the particle's hemostatic activity. In some embodiments, the molar percentage of the PBP, CBP, and/or VBP conjugated to the particle's surface is <5 molar percent. In some embodiments the molar percentage of the PBP, CBP, and/or VBP is between 5% and about 0.5%, between 5% and about 1%, between 5% and about 2%, between 5% and about 3%, between 5% and about 4%, exclusive of 5% and inclusive of the lower range limit. In some embodiments the molar percentage of the PBP, CBP, and/or VBP is between about 4% and about 0.1%, between about 3% and about 0.1%, between about 2% and about 0.1%, between about 1% and about 0.1%, between about 4% and about 0.5%, between about 3% and about 0.5%, between about 2% and about 0.5%, or between about 1% and about 0.5%.

The ratio of VBPs to CBPs conjugated on the particle surface can be about 70:30 to about 30:70 and be adjusted accordingly to maximize adhesion under low-to-high shear conditions. In some embodiments, the ratio of VBP:CPB:PBP can be about 1:1:2 to 1:2:1 to 2:1:1. In some embodiments, the relative molar ratios of PBP:CBP:VBP are 1:5:5. In some embodiments, the relative molar ratios are between about 1:1:1 and about 1:5:1, between about 1:1:1 and about 1:1:5, between about :1:1:1 and about 1:5:5, between about 1:1:1 and about 5:1:1, between about 1:1:1 and about 5:5:1, or between about 1:1:1 and about 5:1:5. In some embodiments, the relative molar ratios of PBP:CBP:VBP are about 2:1:1, about 1:5:5, about 10:5:1, about 10:1:5, about 1:2:1, about 1:1:2, about 10:1:1, about 2:1:0, about 2:0:1, about 1:0:0, about 0:1:0, about 0:0:1, or about 0:1:1, or any ratio between any two of these ratios. It will be appreciated, that other ratios can be used to enhance the particle adherence and activated platelet aggregation.

In some embodiments, the compositions comprising a synthetic platelet described herein, can be formulated and administered to an animal, preferably a human, in need of reducing or slowing blood loss. In other embodiments, the compositions comprising a synthetic platelet described herein, may be formulated and administered to an animal, preferably a human, to facilitate the delivery of a therapeutic agent.

In some embodiments, the synthetic platelets described herein can be provided in a pharmaceutical composition. Such a pharmaceutical composition may consist of a synthetic platelet alone, in a form suitable for administration to a subject, or the pharmaceutical composition may comprise a synthetic platelet and one or more pharmaceutically acceptable carriers, one or more additional ingredients, one or more pharmaceutically acceptable therapeutic agents, bioactive agents, diagnostic agents, or some combination of these. The therapeutic agent may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the therapeutic agent may be combined and which, following the combination, can be used to administer the therapeutic agent to a subject.

As used herein, the term “physiologically acceptable” ester or salt means an ester or salt form of the therapeutic agent which is compatible with any other ingredients of the pharmaceutical composition, which is not deleterious to the subject to which the composition is to be administered.

In some embodiments, the bioactive agent, diagnostic agent, and/or therapeutic agent can be conjugated, encapsulated, and/or contained with the synthetic platelet so that synthetic platelet acts as a delivery vehicle. In other embodiments, the bioactive agent, diagnostic agent, and/or therapeutic agent can be merely contained in a pharmaceutical composition either with or without the synthetic platelets and administered to concurrently with or separately from administration of the synthetic platelets. Selection of a bioactive agent, diagnostic agent, and/or therapeutic agent to be conjugated to or encapsulated within the synthetic platelet is dependent upon the use of the synthetic platelet and/or the condition being treated and the site and route of administration.

Bioactive agents encapsulated by and/or conjugated to the synthetic platelet can include any substance capable of exerting a biological effect in vitro and/or in vivo. Examples of bioactive agents can include, but are not limited to, biologically active ligands, small molecules, proteins, DNA fragments, DNA plasmids, interfering RNA molecules, such as siRNAs, mRNAs, oligonucleotides, and DNA encoding for shRNA. Diagnostic agents can include any substance that may be used for imaging a region of interest (ROI) in a subject and/or diagnosing the presence or absence of a disease or diseased tissue in a subject. Therapeutic agents can refer to any therapeutic or prophylactic agent used in the treatment (including the prevention, diagnosis, alleviation, or cure) of a malady, affliction, condition, disease or injury in a subject. It will be appreciated that the membrane can additionally or optionally include proteins, carbohydrates, polymers, surfactants, and/or other membrane stabilizing materials, any one or combination of which may be natural, synthetic, or semisynthetic.

The methods of treatment using the synthetic platelets described herein include administering a therapeutically effective amount of a synthetic platelet to a subject in need thereof. It should be understood, that the methods of treatment by the delivery of a synthetic platelet include the treatment of subjects that are already bleeding, as well as prophylactic treatment uses in subjects not yet bleeding. In a preferred embodiment, the subject is an animal. In a more preferred embodiment, the subject is a human.

In some aspects, methods of treating a subject having or suspected of having cancer are provided in which the subject is administered a pharmaceutical composition comprising a particle described herein. In some embodiments, the pharmaceutical composition comprises the particle and an anti-cancer therapeutic agent. In some embodiments, the particle encapsulates or is conjugated to the anticancer agent.

In some aspects, methods are provided for preventing or inhibiting platelet aggregation in a subject in need thereof, the method comprising administering to the subject a pharmaceutical composition comprising a fibrinogenic mimetic peptide described herein

The embodiments described herein should in no way be construed to be limited to the synthetic platelets described herein, but rather should be construed to encompass the use of additional synthetic platelets, both known and unknown, that diminish or reduce bleeding or blood loss.

The formulations of the pharmaceutical compositions described herein may be prepared by any method known or hereafter developed in the art of pharmacology. In general, such preparatory methods include the step of bringing a synthetic platelet into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions, which are suitable for ethical administration to humans, it will be understood by the skilled artisan that such compositions are generally for administration to animals of all sorts. Modification of pharmaceutical compositions for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, animals including commercially relevant animals such as cattle, pigs, horses, sheep, cats, and dogs, birds including commercially relevant birds such as chickens, ducks, geese, and turkeys.

Pharmaceutical compositions that are useful in the methods described herein may be administered, prepared, packaged, and/or sold in formulations for parenteral, oral, rectal, vaginal, topical, transdermal, pulmonary, intranasal, buccal, ophthalmic, or another route of administration.

The compositions described herein may be administered via numerous routes, including, but not limited to, parenteral, oral, rectal, vaginal, topical, transdermal, pulmonary, intranasal, buccal, or ophthalmic administration routes. The route(s) of administration will be readily apparent to the skilled artisan and will depend upon any number of factors including the type and severity of the disorder being treated, the type and age of the veterinary or human patient being treated, and the like.

Parenteral administration of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition on or through a surgical incision, by application of the composition on or through a tissue-penetrating non-surgical wound, and the like. In particular, parenteral administration is contemplated to include, but is not limited to, cutaneous, subcutaneous, intraperitoneal, intramuscular, intrasternal, intravenous, and intra-arterial administration.

Formulations of a pharmaceutical composition suitable for parenteral administration comprise the therapeutic agent combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles. Such formulations may further comprise one or more additional ingredients including, but not limited to, suspending, stabilizing, or dispersing agents. In one embodiment of a formulation for parenteral administration, the therapeutic agent is provided in dry (i.e., powder or granular) form for reconstitution with a suitable vehicle (e.g., sterile pyrogen free water) prior to parenteral administration of the reconstituted composition.

Pharmaceutical compositions that are useful in the methods described herein may be administered systemically in oral solid formulations, ophthalmic, suppository, aerosol, topical or other similar formulations. In addition to the compound such as heparin sulfate, or a biological equivalent thereof, such pharmaceutical compositions may contain pharmaceutically acceptable carriers and other ingredients known to enhance and facilitate administration.

The pharmaceutical compositions described herein may also be formulated so as to provide slow, prolonged or controlled release. In general, a controlled-release preparation is a pharmaceutical composition capable of releasing the synthetic platelet at a desired or required rate to maintain constant activity for a desired or required period of time.

A pharmaceutical composition described herein may be prepared, packaged, or sold in bulk, as a single unit dose, or as a plurality of single unit doses. As used herein, a “unit dose” is discrete amount of the pharmaceutical composition comprising a predetermined amount of the activity. The amount of the activity is generally equal to the dosage, which would be administered to a subject or a convenient fraction of such a dosage such as, for example, one-half or one-third of such a dosage.

The relative amounts of the ingredients in a pharmaceutical composition of the invention will vary, depending upon the identity, size, and condition of the subject treated and further depending upon the route by which the composition is to be administered. By way of a non-limiting example, the composition may comprise between 0.1% and 100% (w/w) of the synthetic platelets.

The synthetic platelet compositions described herein may be administered to deliver a dose of between about 1 ng/kg/day and about 100 mg/kg/day. In one embodiment, a dose can be administered that results in a concentration of the synthetic platelets between about 0.01 g/mL and about 625 μg/mL in the blood of a mammal. While the precise dosage administered will vary depending upon any number of factors, including but not limited to, the type of animal, the amount of bleeding being treated, the type of bleeding being treated, the type of wound being treated, the age of the animal and the route of administration. Preferably, the dosage of the synthetic platelet will vary from about 1 μg to about 50 mg per kilogram of body weight of the animal. More preferably, the dosage will vary from about 10 g to about 15 mg per kilogram of body weight of the animal. Even more preferably, the dosage will vary from about 100 g to about 10 mg per kilogram of weight of the animal.

The pharmaceutical compositions may be prepared, packaged, or sold in the form of a sterile injectable aqueous or oily suspension or solution. This suspension or solution may be formulated according to the known art, and may comprise, in addition to the therapeutic agent, additional ingredients such as the dispersing agents, wetting agents, or suspending agents described herein. Such sterile injectable formulations may be prepared using a non-toxic parenterally acceptable diluent or solvent, such as water or 1,3butane diol, for example. Other acceptable diluents and solvents include, but are not limited to, Ringer's solution, isotonic sodium chloride solution, and fixed oils such as synthetic mono or di-glycerides.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

The pharmaceutical composition may be administered to an animal as needed. The pharmaceutical composition may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

### Methods of Lyophilization

In one aspect of the invention, a method is provided for forming a dried composition, the method comprising incubating a composition at a temperature from about 0 to about −210° C. to form a cooled composition; and incubating the cooled composition at a temperature from about 0 to about −60° C. at a pressure from about 0.1 to about 1.0 mTorr, thereby forming a dried composition. In some embodiments, the temperature at which the composition is cooled is between about 0 and about −250° C., about 0 and about −200° C., about 0 and about −150° C., about 0 and about −100° C., about 0 and about −50° C., about −50 and about −250° C., about −100 and about −250° C., about −150 and about −250° C., about −150 and about −250° C., or about −200 and about −250° C. In some embodiments, the cooled composition is subjected to a temperature between about 0 to about −100° C., about 0 to about −90° C., about 0 to about −80° C., about 0 to about −70° C., about 0 to about −60° C., about 0 to about −50° C., about 0 to about −40° C., about 0 to about −30° C., about 0 to about −20° C., about 0 to about −10° C., about −10 to about −100° C., about −20 to about −100° C., about −30 to about −100° C., about −40 to about −100° C., about −50 to about −100° C., about −60 to about −100° C., about −70 to about −100° C., about −80 to about −100° C., or about −90 to about −100° C. In some embodiments, the cooled composition is subjected to a temperature as described above at a pressure of about 0.1 to about 1.0 mTorr. In some embodiments, the cooled composition is subjected to a temperature at a pressure of about 0.1 to about 0.9 mTorr, about 0.1 to about 0.8 mTorr, about 0.1 to about 0.7 mTorr, about 0.1 to about 0.6 mTorr, about 0.1 to about 0.5 mTorr, about 0.1 to about 0.4 mTorr, about 0.1 to about 0.3 mTorr, about 0.1 to about 0.2 mTorr, about 0.2 to about 0.9 mTorr, about 0.3 to about 0.9 mTorr, about 0.4 to about 0.9 mTorr, about 0.5 to about 0.9 mTorr, about 0.6 to about 0.9 mTorr, about 0.7 to about 0.9 mTorr, or about 0.8 to about 0.9 mTorr.

In some embodiments, the lipid particles in the dried composition have a diameter from about 1 nm to about 1000 nm. In some embodiments, the lipid particles in the dried composition have a diameter between about 100 nm to about 1000 nm, about 100 nm to about 900 nm, about 100 nm to about 800 nm, about 100 nm to about 700 nm, about 100 nm to about 600 nm, about 100 nm to about 500 nm, about 100 nm to about 400 nm, about 100 nm to about 300 nm, about 100 nm to about 200 nm, about 100 nm to about 100 nm, about 200 nm to about 1000 nm, about 300 nm to about 1000 nm, about 400 nm to about 1000 nm, about 500 nm to about 1000 nm, about 600 nm to about 1000 nm, about 700 nm to about 1000 nm, about 800 nm to about 1000 nm, or about 900 nm to about 1000 nm.

In some embodiments, the lipid particles in the dried composition have an average diameter from about 1 nm to about 1000 nm. In some embodiments, the lipid particles in the dried composition have an average diameter between about 100 nm to about 1000 nm, about 100 nm to about 900 nm, about 100 nm to about 800 nm, about 100 nm to about 700 nm, about 100 nm to about 600 nm, about 100 nm to about 500 nm, about 100 nm to about 400 nm, about 100 nm to about 300 nm, about 100 nm to about 200 nm, about 100 nm to about 100 nm, about 200 nm to about 1000 nm, about 300 nm to about 1000 nm, about 400 nm to about 1000 nm, about 500 nm to about 1000 nm, about 600 nm to about 1000 nm, about 700 nm to about 1000 nm, about 800 nm to about 1000 nm, or about 900 nm to about 1000 nm.

In some embodiments, the lipid particles in the dried composition have a spherical morphology. In some embodiments, the lipid particles in the dried composition have a polydispersity index from about 0.1 to about 0.5, about 0.2 to about 0.5, about 0.3 to about 0.5, about 0.4 to about 0.5, about 0.1 to about 0.4, about 0.1 to about 0.3, or about 0.1 to about 0.2. In some embodiments, the lipid particles have a polydispersity index no greater than 0.1, 0.2, 0.3, 0.4, or 0.5. In some embodiments, the lipid particles have a polydispersity index no greater than 0.3.

In some embodiments, the lipid particles in the dried composition have a net positive or net negative zeta potential. In some embodiments, the lipid particles in the dried composition have a shelf-life of at least 2 years at ambient conditions. For example, in some embodiments, the lipid particles in the dried composition have a shelf-life of at least 2 years at storage conditions between −20° C. to 4° C. or at storage conditions up to 50° C. In some embodiments, the lipid particles in the dried composition retain loading of one or more therapeutic agents.

The following examples are put forth so as to provide those of ordinary skill in the art with a complete disclosure and description of how to make and use the assay, screening, and therapeutic methods of the invention, and are not intended to limit the scope of what the inventors regard as their invention.

### Examples

### Example 1: Methods for Lyophilizing Synthetic Platelets

### Summary

To develop a method of lyophilizing synthetic platelets, the parameters for freezing, primary drying, and secondary drying based on lyophilization cake appearance and reconstitution ease were first explored. Next, the lyophilization volumes and lipid concentrations based on size and charge measurements by an Anton Paar Litesizer™ were examined. Then, the composition of the lyophilization buffer was studied, including lyoprotectant and its concentration, buffering agent and concentration, and buffer pH, which were determined by both physicochemical and functional characteristics. The lyophilization parameters were evaluated based on the physical and functional characterization of the lyophilized synthetic platelets for size by dynamic light scattering (DLS), charge by zeta potential, morphology and size by cryogenic tunneling electron microscopy (cryo-TEM), and platelet binding functionality by flow cytometry. Reproducibility of the method was evaluated with a target percent coefficient of variation (% CV) below 25% across batches of lyophilization.

### Materials and Methods

### Synthetic Platelet Formulation

Synthetic platelet formulation, Particle1, with Cy5 dye containing platelet binding, collagen. Binding, and vWF binding peptides (PBP, CBP, and VBP, respectively) was manufactured using the standard lipid film rehydration method for liposome preparation, followed by extrusion through 200 nm followed by 100 nm pore size filters. Various lyoprotectant and buffering agents were added during the lipid film rehydration step, and in some cases additional lyoprotectant was added after extrusion. The individual reagent components for Particle1 and their relative amounts are summarized in Table 1 and Table 2. Equipment and other supplies used during the manufacture and characterization of the lyophilized Particle1 are summarized in Table 3 and Table 4. Triplicate batches with each lyophilization group were produced to obtain an inter-lot mean and standard deviation. In addition, all samples were analyzed in triplicate to obtain an intra-lot mean value and standard deviation. The inter-lot standard deviation was used to verify the sample size using the power analysis equation and to justify the triplicate lot sample size.

TABLE 1
Lyophilized Particle1 Materials

| Reagent | Molecular Weight (Da) | Mole (%) | Vendor | Catalog # | Lot # |
|---|---|---|---|---|---|
| DSPE- PEG2K-PBP | 4001.0 | 1 | Bachem | 4148311 | 1000043016 |
| DSPE- PEG2K-VBP | 4979.0 | 0.5 | Bachem | 4148310 | 1000040223 |
| DSPE- PEG2K-CBP | 4933.8 | 0.5 | Bachem | 4148308 | 1000040222 |
| DSPC | 790.2 | 46.5 | Avanti | 850365 | 5353PQB160 |
| Cholesterol | 386.7 | 45.0 | Fisher Scientific | ICN10138201 | SLBR4060V |
| DSPE-mPEG1k | 1788.3 | 5.5 | Avanti | 880730 | 880720P-200 MG-C-025 |
| DSPE-Cy5 (Dye) | 1266.2 | 1.0 | Avanti | 810345G-5 mg | 810345C-5 mg-A-010 |
| Trehalose | 342.3 | N/A | VWR | 10118-236 | 18D2756862 |
| Dextrose | 180.2 | N/A | VWR | JT1910-1 | TBD |
| Sucrose | 342.3 | N/A | VWR | JT4074-1 | TBD |
| HP-β-CD | 1541.5 | N/A | Sigma Aldrich | H107-100G | TBD |

TABLE 2
Lyophilization Buffer Composition
in Various Particle1 Formulations

| | | | Lyo | |
|---|---|---|---|---|
| Buffer | Buffer Agent | Lyo | Protectant | Buffer |
| Agent | Conc. (mg/mL) | Protectant | Conc. % (w/v) | pH |
| Tris | 3 | Sucrose | 10 | 7.0 |
| | | | | 9.0 |
| | | | 5 | 7 |
| | | | | 9.0 |
| | | Dextrose | 10 | 7 |
| | | | | 9.0 |
| | | HP-β-CD | 10 | 7 |
| | | | | 9.0 |
| HEPES | 2 | Sucrose | 10 | 7 |
| | | | | 5.5 |
| | | Dextrose | 10 | 7 |
| | | | | 5.5 |
| | | HP-β-CD | 10 | 7 |
| | | | | 5.5 |
| | 4 | Dextrose | 10 | 5.5 |
| | | | 5 | 5.5 |
| | | | 1 | 5.5 |
| | | | 5% Out/1% In | 5.5 |
| | | Trehalose | 10 | 5.5 |
| | | | 5 | 5.5 |
| | | | 1 | 5.5 |
| | | | 5% Out/1% In | 5.5 |

TABLE 3
Equipment

| Equipment | Manufacture | Model # | Serial # |
|---|---|---|---|
| Lyovapor L-200 Pro | Buchi | L-200 Pro | 1500029540 |
| Rotavapor R-200 | Buchi | R-200 | 0400023948 |
| Extruder | LIPEX | 10-mL | 002179 |
| ZetaSizer | Anton Paar | Litesizer 500 | 82870232 |
| Fluorescence Plate | Synergy | Synergy Mx | 255437 |
| Reader | | | |
| Prism Software | GraphPad | Version 8.4.3 | GPS-15552314 |
| | | (471) | |
| 8-Channel 200 μL | VWR | 89079-948 | A53830065 |
| Micropipette | | | |
| Tecnai T12 Cryo-TEM | Tecnai | T12 | 12TN166B/ |
| | | | D1159 |
| ImageJ.JS Software | NIH | Version 1.53f | N/A |
| Flow Cytometer | BD | Accuri C6 | SN 5247 |

TABLE 4
Other Supplies

| Supplies | Vendor | Catalog # | Lot # |
|---|---|---|---|
| 200 μL Pipette Tips | VWR | 470146-178 | N/A |
| Omega Zeta Potential Cuvettes | Anton | 225288 | 80417675 |
| | Paar | | |
| Lacey Carbon Coated Copper | Ted | 01890-F | 130818 |
| TEM Grids | Pella | | |

### Lyophilization Cycle Development

Initially, lyophilization parameters including freezing temperature, primary drying temperature and pressure, and secondary drying temperature and pressure were tested for lyophilized synthetic platelet cake appearance, yield, and ease of reconstitution. The lyophilized synthetic platelet cake results from this study are summarized in Table 5. Insufficient freezing resulted in sample boiling and loss of product, secondary drying temperatures above room temperature resulted in lyophilized synthetic platelet cake that was difficult to reconstitute, and using a temperature ramp in secondary drying resulted in lyophilized synthetic platelet cake that was easier to reconstitute. Lyophilization runs that implemented a precooling of the lyophilizer shelf to achieve a −45° C. to 25° C. temperature ramp allowed for a distinct primary and secondary drying phase. For primary and secondary drying phases, both were set to a lyophilizer condenser temperature of −45° C. and a vacuum of 0.2 mTorr for 960 min. The primary drying phase for 1 hour was achieved by pre-cooling the lyophilizer shelf temperature to −60° C. and allowing it to naturally reach 0° C. after about 1 hour of natural heat transfer with the lyophilizer system for an approximate rate of +1° C./min. This precooling of the lyophilizer shelf was accomplished by removing the shelf and placing it in the −80° C. freezer for 2 hours and then placing in back in the Lyovapor L-200 Pro (TL036) lyophilizer just prior to loading the shell frozen samples. Once the shelf temperature reached 0° C. and continued to warm to ambient (−25° C.), this phase was considered secondary drying. Secondary drying is intended to remove all residual water by getting water molecules to sublimate faster with increased temperature (Iyer et al., J. Pharmaceutical Sciences (2016) 105(5): 1684-1692). This lyophilization process was then repeated for five separate runs to demonstrate reproducibility of the process.

TABLE 5
Lyophilization Cycle Conditions Evaluated

| | | Primary Drying | Secondary Drying | |
|---|---|---|---|---|
| Freeze Temp | | Temp & Pressure | Temp & Pressure | Lyophilized Synthetic Platelet Cake |
| (° C.) | | (° C., mTorr) | (° C., mTorr) | Appearance and Observations |
| −80° | C. | −45° C., | 25° C., | Rigid Foam Cake, Loss of Cake via |
| | | 0.700 mTorr | 0.200 mTorr | Boiling, |
| | | | | Mostly Reconstitute 5 min |
| −196° | C. | −45° C., | 25° C., | Rigid Foam Cake, |
| | | 0.700 mTorr | 0.200 mTorr | Mostly Reconstitute 5 minutes |
| −196° | C. | −45° C., | 4-hr Ramp: −45° | Crystalline Cake, |
| | | 0.700 mTorr | C. to 30° C. | Does Not Reconstitute Easily |
| | | | 6-hr Hold: 30° C. | |
| | | | 3-hr Ramp; 30 to 35° C. | |
| | | | 3-hr Hold: 35° C. | |
| | | | 0.200 mTorr | |
| −196° | C. | −45° C., | 25° C., | Rigid Foam Cake, Mostly |
| | | 0.200 mTorr | 0.200 mTorr | Reconstitute 5 min |
| −196° | C. | −45° C., | 2-hr Ramp: −45° | Fluffier Foam Cake, Reconstitute 5 |
| | | 0.200 mTorr | C. to 25° C. | min |
| | | | 16-hr Hold: 25° C. | |
| | | | 0.200 mTorr | |

### Lyophilization Volume and Concentration

Freezing volume and concentration for lyophilization samples were determined by shell-freezing various concentrations and volumes of SP in 10% (w/v) trehalose in 4 mg/mL HEPES as shown in Table 6. The shell-freezing was achieved by submerging the glass vial containing SP at approximately a 450 angle into liquid nitrogen (−196° C.) for 5 minutes while rotating at approximately 30 rpm (Wang et al., (2019) Advanced Drug Delivery Reviews 151-152: 56-71). Each lyophilized freezing group was resuspended in cell culture grade water and analyzed for size and charge to determine the freezing concentration and volume.

TABLE 6
Size and Charge Results at Various Lyophilization Volumes and Concentrations

| | | | | Mean | Mean | Mean | |
|---|---|---|---|---|---|---|---|
| Freezing | Freezing | Freezing | | Zeta | Hydrodynamic | Diameter | |
| Container | Volume | Conc. | | Potential | Diameter | Intensity | |
| Volume (mL) | (mL) | (mg/mL) | Lyophilization | (mV) | (nm) | (nm) | PDI |
| 50 | 30 | 0.2 | Pre | −31 | 160 | 153 | 0.18 |
| | | | Post | −25 | 483 | 393 | 0.28 |
| 50 | 12 | 0.5 | Pre | −31 | 160 | 153 | 0.18 |
| | | | Post | −29 | 378 | 232 | 0.33 |
| 10 | 6 | 1.0 | Pre | −31 | 160 | 153 | 0.18 |
| | | | Post | −25 | 5654 | 3011 | 0.25 |
| 10 | 3 | 2.0 | Pre | −31 | 160 | 153 | 0.18 |
| | | | Post | −26 | 1917 | 2841 | 0.24 |

### Physicochemical and Functional Characterization of Lyophilized Particle1

### Particle Diameter by Litesizer

Particle1 in each lyoprotectant and rehydration buffer combinations was diluted in their respective buffers to a 0.1 mg/mL solution for Litesizer analysis for particle size measurements using a refractive index of 1.34 and a viscosity of 0.00099 Pa*s.

### Charge by Zeta Potential

Particle1 in each lyoprotectant and rehydration buffer combination was diluted in their respective buffers to a 0.1 mg/mL solution for Litesizer analysis for Zeta Potential analysis measured in millivolts (mV) using a refractive index of 1.34 and a viscosity of 0.00099 pa*s.

Morphology and Size by cryo-TEM

Cryo-TEM analysis of Particle1 in the various lyoprotectant and buffer concentrations were conducted for morphology evaluation with plunge frozen cryo-TEM samples. Samples were prepared by diluting Particle1 to 0.5 mg/ml in cell culture grade water, adsorbed onto glow-discharged lacey carbon-coated copper grids for 1 minute, blotted with filter paper for 8 seconds at force of 10, and plunge frozen in liquid ethane (below the devitrification temperature of −137° C.). Samples were imaged between 9,000×-30,000× magnification. Diameters of the particles in cryo-TEM images were measured using the online software ImageJ.

### Functionality by Flow Cytometry

Synthetic platelet functionality of binding to activated platelets was evaluated using flow cytometry. In brief, platelet rich plasma (PRP) from healthy human donors was diluted by 2-fold with Tyrode's buffer (137 mmol/L NaCl, 12 mmol/L NaHCO₃, 2.0 mmol/L KCl, 0.3 mmol/L Na₂HPO₄, 1 mmol/L MgCl₂, 5 mmol/L HEPES, 5 mmol/L glucose, pH 7.3) and supplemented with 0.03 units/mL apyrase and centrifuged at 100×g for 15 minutes at 25° C. to pellet any contaminating red and white blood cells.

PRP was supplemented with 1 g/mL prostacyclin for 5 minutes and centrifuged at 600×g for 15 minutes at 25° C. for platelet washing.

The platelet pellet was gently resuspended in Tyrode's buffer and allowed to equilibrate on the benchtop for 20-30 minutes prior to flow cytometry staining.

The washed platelets were aliquoted into triplicate flow cytometry tubes, incubated at 25° C. with FITC anti-CD62P, 5 M of TRAP agonist, and each Cy5-labeled synthetic platelet formulation for 20 min.

Platelets were read on a flow cytometer until 50,000 counts were measured per sample with gating for platelet populations using side scatter (SSC) on the y-axis and forward scatter (FCS) on the x-axis all in log plots. Degree of activation in the platelet population was evaluated via FITC staining, and particle binding to platelets was evaluated via Cy5 fluorescence levels and % positive platelets.

### Functionality in a Thrombocytopenic Mouse Model

For functionality in a thrombocytopenic (TCP) mouse model, platelet counts from wild-type C57/BL6J mice were achieved via a retro orbital (RO) blood draw of 0.1 mL for platelet counting via the HemaVet 950. Dosage calculations were performed, and mice were injected intraperitoneally with anti-CD42b (anti-GPIba) antibody at 0.2 g/g. 18 hours after antibody injection, platelet counts were again obtained to monitor for induction of thrombocytopenia (~75% average reduction in platelet counts). SP dosing was administered at 0.1, 1.0, and 10.0 mg/kg. Fifteen (15) minutes after treatment administration, transect the tails of these mice 1 mm from the tip with a sharp surgical blade, and immerse in 1200 μL of warm (37° C.) saline. Record the time taken for bleeding to stop (bleeding time). Also, record the blood loss volume using the hemoglobin reagent method.

### Data Analysis

All unique sample configurations were performed in at least triplicate in order to obtain a mean value and standard deviation (Equation 1). This standard deviation (σ) along with Z of 1.96 (95% confidence) and a margin of error of 25% (E) was used to calculate the appropriate sample sizes (n) using Cochrane's power analysis equation as shown in Equation 3 for method validation. Statistical significance was determined using a one-way ANOVA analysis (GraphPad Prism). All results with p-values <0.05 are considered significant.

$$Standard Deviation = \sqrt{\frac{\sum {( X - Mean )}^{2}}{n - 1}} \qquad ( Equation 1 )$$

$$n = {( Z σ / E )}^{2} \qquad ( Equation 3 )$$

### Results

### Size and Charge of Particle1 in Various Lyophilization Buffers

Particle1 formulations with HEPES and TRIS buffers containing various lyoprotectants including dextrose, sucrose, trehalose, and HP-β-CD were examined for size and charge after lyophilization/reconstitution by the Litesizer 500. The results for Particle1 formulations in HEPES and TRIS are summarized in Table 7 and Table 8, respectively.

TABLE 7
Size and Charge of Particle1 in HEPES Buffers Containing Various Lyoprotectants

| | | Lyo | | | | Mean | |
|---|---|---|---|---|---|---|---|
| | | Protectant | | Zeta | Hydrodynamic | Intensity | |
| HEPES Conc. | Lyo | Conc. | Buffer | Potential | Diameter | Diameter | |
| (mg/mL) | Protectant | (%, w/v) | pH | (mV) | (nm) | (nm) | PDI |
| 4 | Dextrose | 10 | 5.5 | −16 | 5651 | 426 | 0.37 |
| | | 5 | 5.5 | −35 | 350 | 129 | 0.26 |
| | | | | −29 | 85347 | 292 | 0.38 |
| | | 1 | 5.5 | −7 | 79187 | 300 | 1.37 |
| | | 5% Out/1% In | 5.5 | 0 | 47797 | 1126 | 0.50 |
| | Trehalose | 10 | 5.5 | −6 | 98697 | 147 | 0.45 |
| | | | | −31 | 241 | 215 | 0.23 |
| | | | | −30 | 646 | 483 | 0.2 |
| | | 5 | 5.5 | −48 | 48000 | 154 | 0.41 |
| | | | | −28 | 68087 | 144 | 0.4 |
| | | 1 | 5.5 | 0 | 16203 | 0.66 | 0.39 |
| | | 5% Out/1% In | 5.5 | −6 | 59966 | 58 | 0.48 |
| 2 | Sucrose | 10 | 7.0 | −6 | 1,239 | 256 | 0.28 |
| | | 10 | 5.5 | −6.4 | 736 | 196 | 0.24 |
| | Dextrose | 10 | 7.0 | −1.9 | 94,000 | 209 | 0.43 |
| | | 10 | 5.5 | −15 | 63,000 | 602 | 0.6 |
| | HP-β-CD | 10 | 7.0 | −5 | 208 | 163 | 0.31 |
| | | | | −24 | 147 | 148 | 0.27 |
| | | | | −12 | 114 | 115 | 0.23 |
| | | | | −9 | 203 | 198 | 0.23 |
| | | | | −10 | 137 | 151 | 0.27 |
| | | | | −8 | 188 | 191 | 0.24 |
| | | | | −8 | 207 | 170 | 0.23 |
| | | | | −9 | 163 | 134 | 0.18 |
| | | 10 | 5.5 | −12 | 127 | 120 | 0.23 |
| | | | | N/A | 218 | 216 | 0.25 |
| | | | | −26 | 186 | 164 | 0.26 |
| | | | | −17 | 92 | 85 | 0.26 |

TABLE 8
Size and Charge of Particle1 in TRIS Buffers Containing Various Lyoprotectants

| | | Lyo | | | | Mean | |
|---|---|---|---|---|---|---|---|
| TRIS | | Protectant | | Zeta | Hydrodynamic | Intensity | |
| Conc. | Lyo | Conc. | Buffer | Potential | Diameter | Diameter | |
| (mg/mL) | Protectant | (%, w/v) | pH | (mV) | (nm) | (nm) | PDI |
| 3 | Sucrose | 5 | 7.0 | −3.8 | 60,000 | 207 | 0.28 |
| | | 5 | 9.0 | −3.3 | 811 | 280 | 0.36 |
| | Dextrose | 10 | 7.0 | −7.2 | 21,907 | 749 | 0.36 |
| | | 10 | 9.0 | −20.7 | 161,625 | 619 | 0.34 |
| | HP-β-CD | 10 | 7.0 | −1.8 | 152 | 148 | 0.24 |
| | | | 7.0 | −9.2 | 156 | 133 | 0.31 |
| | | | 7.0 | −4.9 | 132 | 120 | 0.25 |
| | | | 7.0 | −9 | 277 | 267 | 0.25 |
| | | | 7.0 | −6 | 210 | 248 | 0.25 |
| | | | 7.0 | −6 | 184 | 171 | 0.22 |
| | | | 7.0 | −8 | 116 | 116 | 0.22 |
| | | | 7.0 | −7 | 111 | 109 | 0.11 |
| | | 10 | 9.0 | −5.2 | 140 | 141 | 0.25 |
| | | | 9.0 | −15 | 187 | 162 | 0.12 |
| | | | 9.0 | −17 | 208 | 196 | 0.23 |
| | | | 9.0 | −8 | 158 | 169 | 0.23 |

TABLE 9
Size and charge measurements for five repeat batches of lyophilized
Particle1 with 10% (w/v) HP-β-CD and 2 mg/mL HEPES pH 7.0 Buffer

| | Pre-lyophilized Particle1 | | | | | Lyophilized Particle1 | | | | |
|---|---|---|---|---|---|---|---|---|---|---|
| | Hydrodynamic | Intensity | Intensity | | Zeta | Hydrodynamic | Intensity | Intensity | | Zeta |
| | Diameter | Diameter | D90 | | Potential | Diameter | Diameter | D90 | | Potential |
| Lot# | (nm) | (nm) | (nm) | PDI | (mV) | (nm) | (nm) | (nm) | PDI | (mV) |
| 1 | 254 | 174 | 247 | 0.31 | −12 | 114 | 115 | 171 | 0.24 | −12 |
| 2 | 201 | 157 | 236 | 0.32 | −9 | 137 | 151 | 242 | 0.27 | −10 |
| 3 | | | | | | 187 | 190 | 288 | 0.24 | −8 |
| 4 | 258 | 200 | 347 | 0.24 | −10 | 207 | 170 | 321 | 0.23 | −8 |
| 5 | | | | | | 163 | 134 | 220 | 0.18 | −9 |
| Mean | 237 | 177 | 277 | 0.29 | −10 | 162 | 152 | 248 | 0.23 | −9 |
| % CV (%) | 13% | 12% | 22% | 15% | −15% | 23% | 19% | 24% | 14% | −18% |
| % Change | | | | | | −32% | −14% | −10% | −20% | −9% |
| Post-Lyo (%) | | | | | | | | | | |

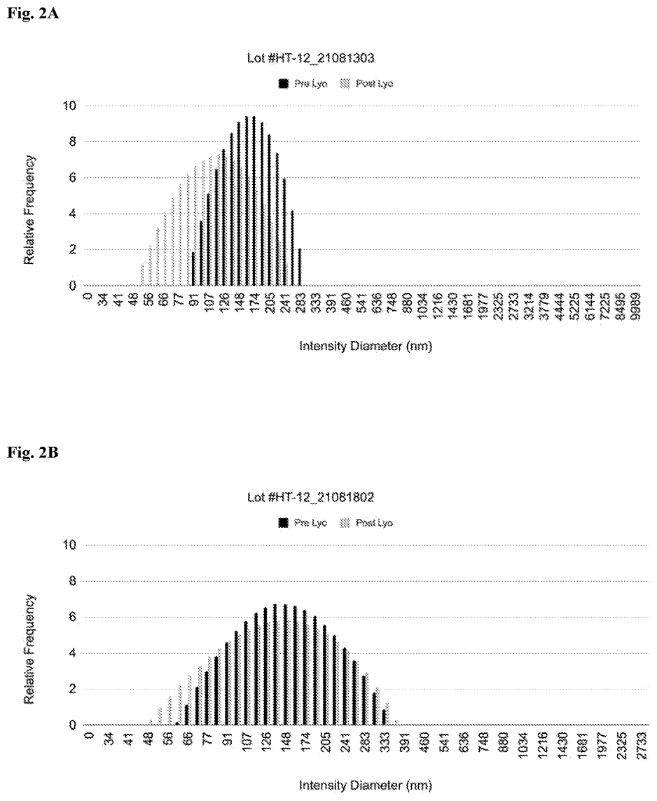

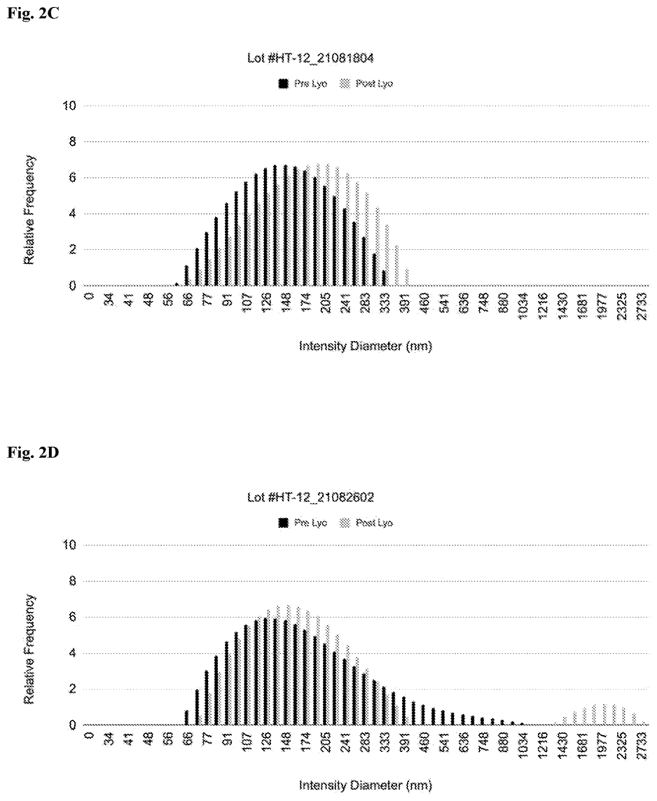

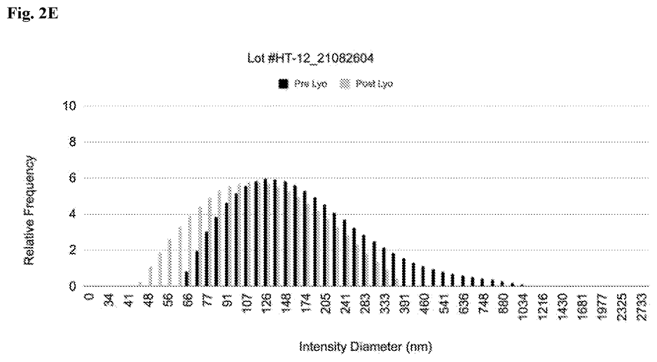

Over the course of the five lyophilization batches, Particle1 in 2 mg/mL HEPES+10% (w/v) HP-β-CD (pH 7) buffer was highly reproducible with % CV for all size parameters and charge below the 25% acceptance criteria as shown in Table 9. The intensity diameter histograms for pre- and post-lyophilized synthetic platelets were overlapping or improved after lyophilization (FIGS. 2A-2E).

### Morphology and Size by Cryo-TEM

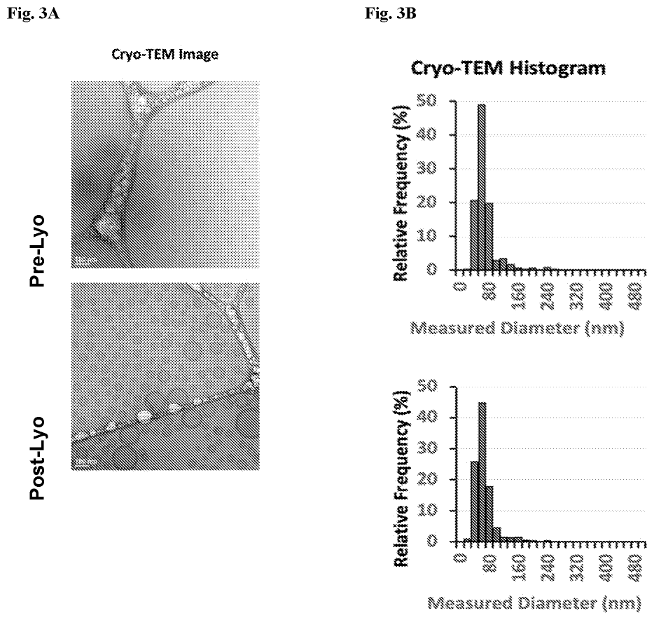

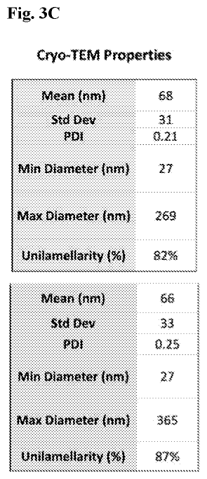

Cryo-TEM was utilized to characterize the morphology and size of Particle1. After lyophilization/reconstitution, size, spherical morphology and unilamellarity were retained as shown in FIGS. 3A-3C.

Physicochemical Stability under Storage at Various Temperatures

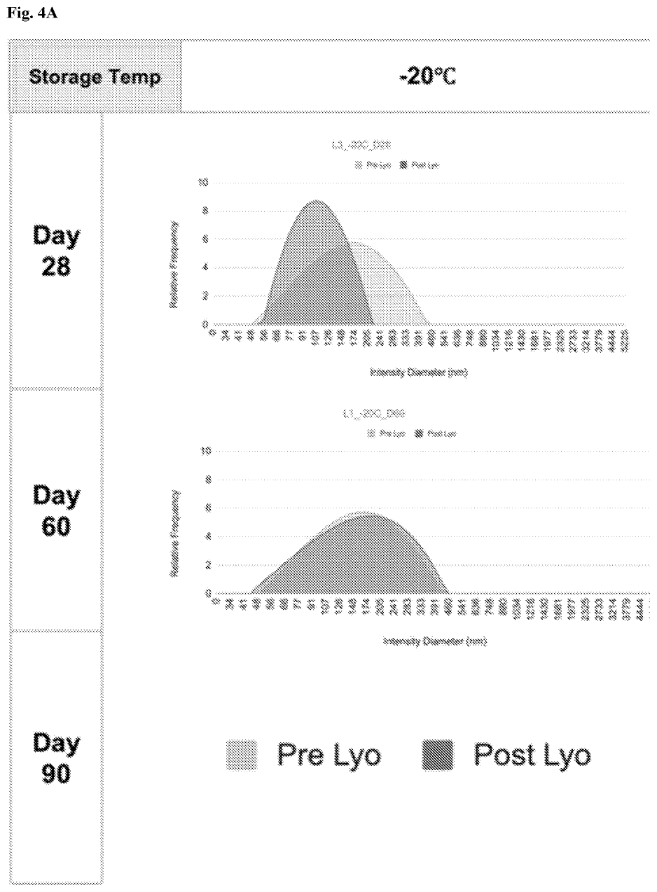

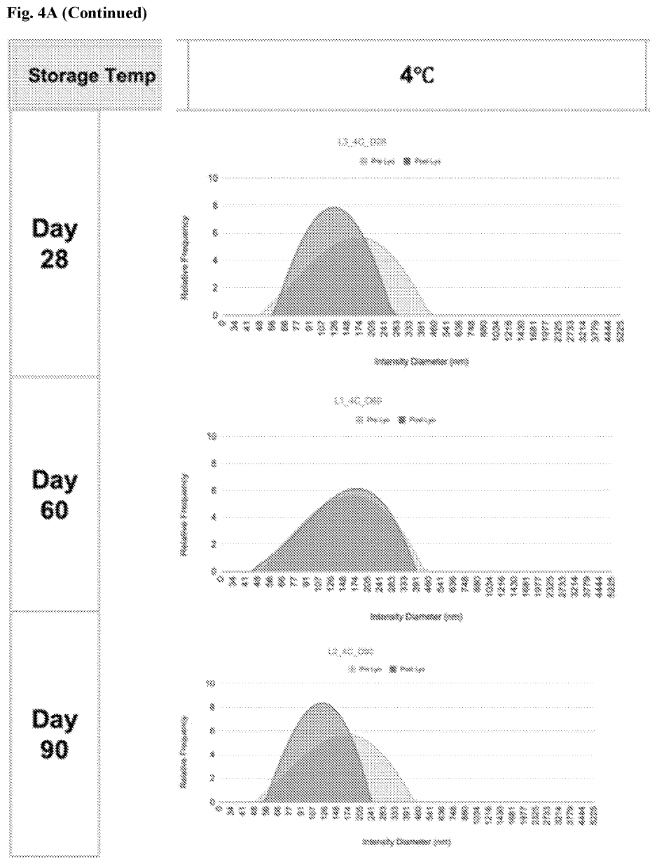

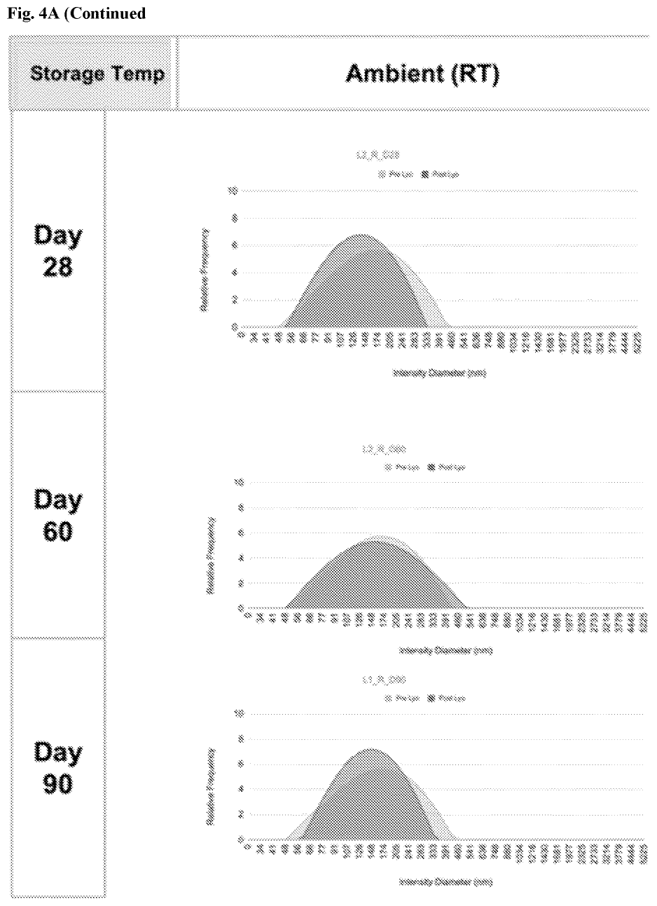

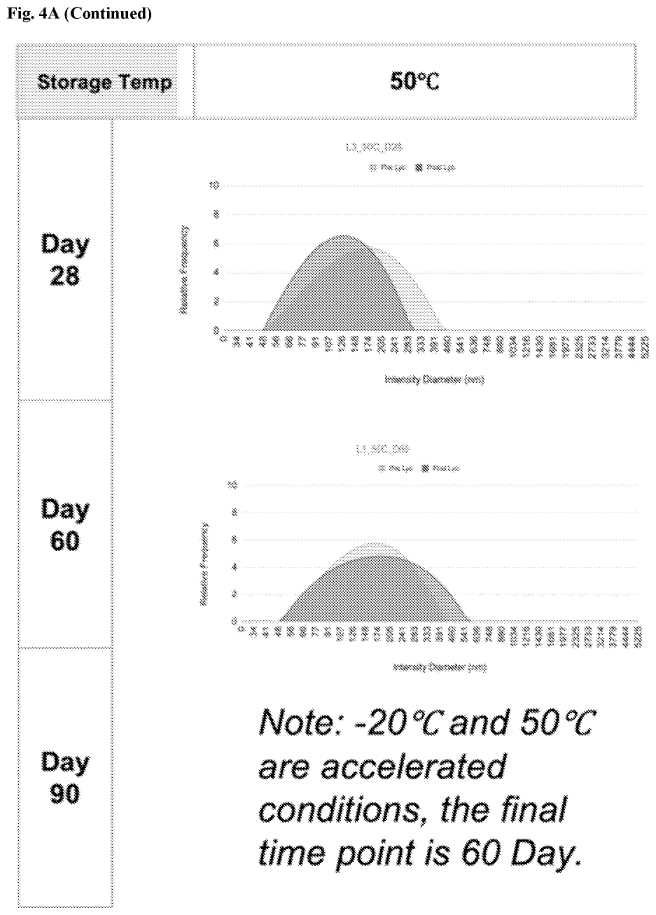

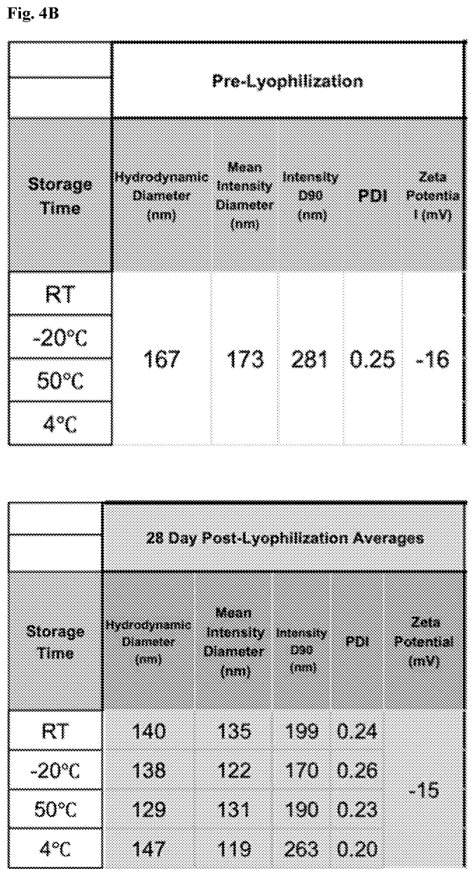

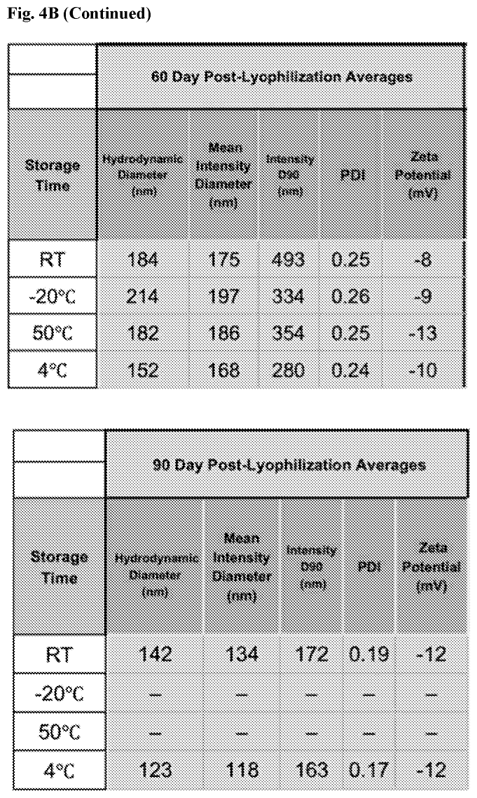

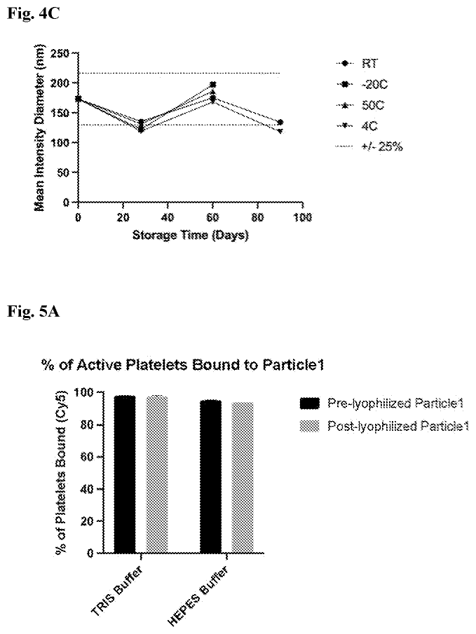

The physicochemical stability of Particle1 with lyophilization buffer 2 mg/mL HEPES+10% (w/v) HP-β-CD (pH 7) with was evaluated under a range of storage temperatures, namely, −20° C., 4° C., room temperature (−25° C.), and 50° C. for up to 3 months. Particle1 was stored as a lyophilized powder at the aforementioned temperatures, and particle size distribution and zeta potential were analyzed using the Litesizer upon rehydration with water at 28 days, 60 days and 90 days. FIG. 4A-C shows that size and charge of Particle1 were maintained within +/−25% of pre-lyophilized Particle1 values, suggesting stability under all storage conditions tested.

### Active Platelet Binding Functionality by Flow Cytometry

The active platelet binding functionality of Particle1 with the lyophilization buffers 2 mg/mL HEPES or 3 mg/mL Tris+10% (w/v) HP-β-CD (pH 7) was evaluated by flow cytometry. The percentage of activated platelets bound to Particle1 was maintained with both the HEPES and Tris post-lyophilized HP-β-CD buffer groups in comparison to pre-lyophilized Particle 1 as shown in FIG. 5A. Similarly, the mean Cy5 fluorescence from platelet bound post-lyophilized Particle1 for both the HEPES and Tris HP-β-CD buffer groups was maintained with comparison to pre-lyophilized Particle as shown in FIG. 5B. Thus, the active platelet binding functionality of lyophilized synthetic platelets was maintained after lyophilization.

### Hemostatic Functionality in a Thrombocytopenic Mouse Model

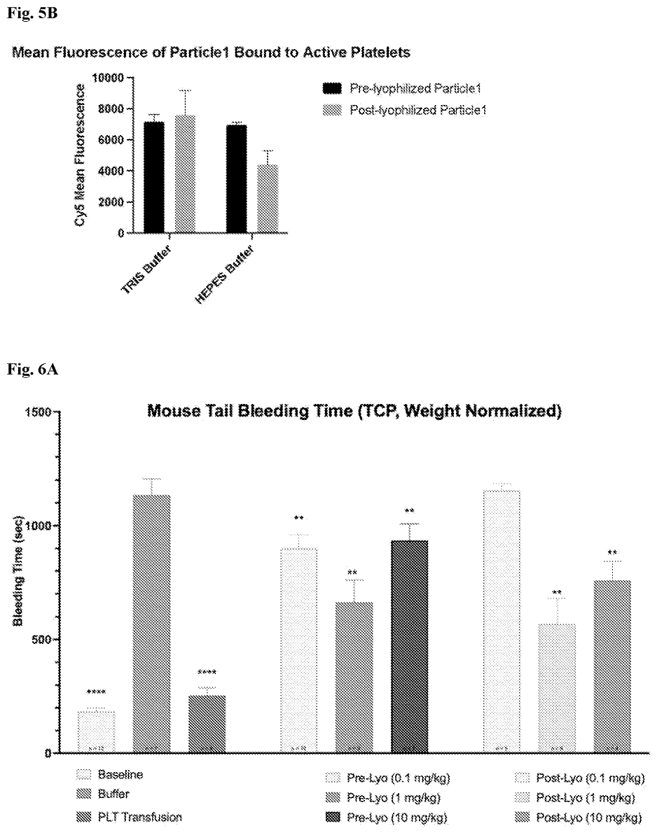

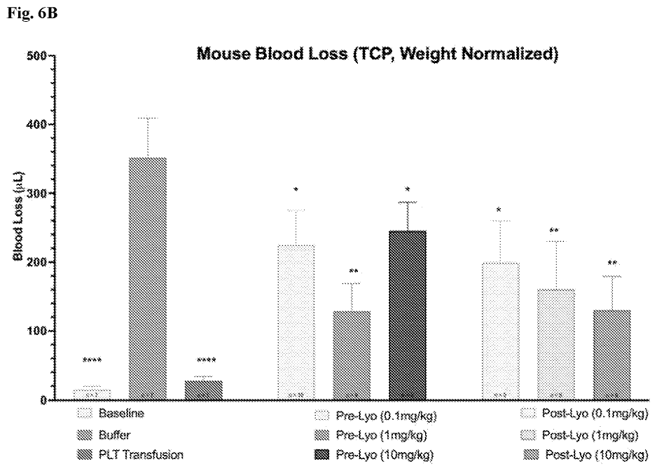

The hemostatic functionality of Particle1 with the lyophilization buffer 2 mg/mL HEPES +10% (w/v) HP-β-CD (pH 7) was evaluated in a thrombocytopenic (TCP) mouse model. The bleed time of TCP mice administered Post-Lyo Particle1 at 1 mg/kg and 10 mg/kg was comparable to Pre-Lyo Particle1 at 1 mg/kg and improved compared at 10 mg/kg as shown in FIG. 6A. Similarly, the blood loss (μL) of TCP mice administered Post-Lyo Particle1 at 1 mg/kg and 10 mg/kg was comparable to Pre-Lyo Particle1 at 1 mg/kg and improved compared at 10 mg/kg as shown in FIG. 6B. Thus, the hemostatic functionality of lyophilized synthetic platelets is maintained after lyophilization.

### Lyo-Cake Reconstitution Study

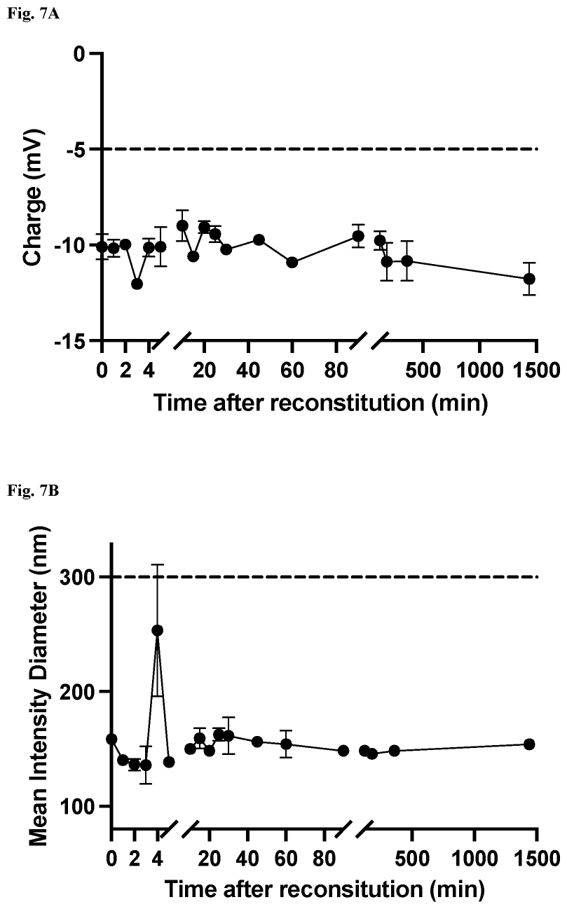

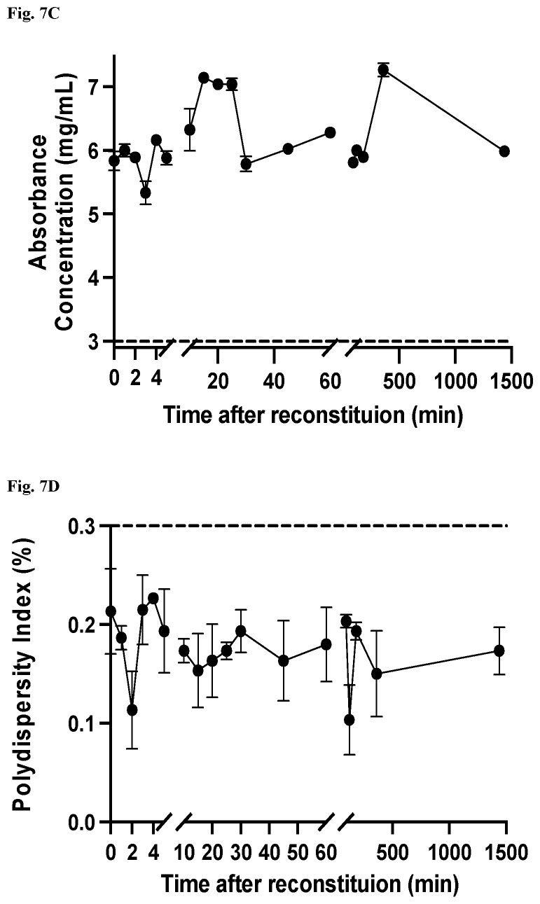

The purpose of this study was to evaluate the physicochemical and visual properties of lyophilized Particle1 in 2 mg/mL HEPES+10% (w/v) HP-β-CD (pH 7) after reconstitution in water over various time points. FIG. 7A shows zeta potential, FIG. 7B shows mean intensity diameter, FIG. 7C shows particle concentration, and FIG. 7D shows polydispersity index of lyophilized Particle1 after reconstitution over time. All parameters were within acceptable limits (represented as dotted lines) at all time points after reconstitution. No visible aggregates were observed in any of the samples.

### CONCLUSIONS

In conclusion, we investigated both the lyophilization method and buffer for maintaining the size range and functionality of synthetic platelets after lyophilization. This buffer was able to retain the size range of 50 nm to 200 nm upon immediate reconstitution or upon storage at a variety of temperatures between −20-50° C. for up to 3 months while also maintaining its ability to bind to active platelets via flow cytometry and reduce bleeding in a thrombocytopenic mouse model. In addition, this lyophilization process has been validated and shown to be highly reproducible with % CV well below 25%.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences that reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed:

1. A composition comprising a plurality of lipid particles and a liquid component, wherein the liquid component comprises water and a cyclodextrin at a concentration from about 1% (w/v) to about 20% (w/v) of the liquid component, wherein each lipid particle comprises a lipid and a peptide conjugate, wherein the peptide of the peptide conjugate is associated with the external surface of the lipid particle.

2. The composition of claim 1, wherein the cyclodextrin is hydroxypropyl-β-cyclodextrin (HP-β-CD), hydroxypropyl-γ-cyclodextrin, hydroxypropyl-α-cyclodextrin, and/or hydroxypropyl cyclodextrin.

3. The composition of claim 1, wherein the cyclodextrin is hydroxypropyl-β-cyclodextrin (HP-β-CD).

4. The composition of any one of claims 1-3, further comprising 4-(2-hydroxyethyl)-1-piperazine ethane sulfonic acid (HEPES) at a concentration from about 0.1% (w/v) to about 0.5% (w/v) of the lipid component, wherein the pH of the liquid component is from about 5 to about 9.

5. The composition of any one of claims 1-3, wherein the composition does not comprise 4-(2-hydroxyethyl)-1-piperazine ethane sulfonic acid (HEPES).

6. The composition of any one of claims 1-5, further comprising tris(hydroxymethyl)aminomethane (tris) at a concentration from about 0.1% (w/v) to about 0.5% (w/v) of the liquid component.

7. The composition of any one of claims 1-5, wherein the composition does not comprise tris(hydroxymethyl)aminomethane (tris).

8. The composition of any one of claims 1-7, further comprising sodium chloride at a concentration from about 0.1% (w/v) to about 1% (w/v) of the liquid component.

9. The composition of any one of claims 1-7, wherein the composition does not comprise sodium chloride.

10. The composition of any one of claims 1-9, further comprising ammonium sulfate at a concentration from about 0.11% (w/v) to about 0.5% (w/v) of the liquid component.

11. The composition of any one of claims 1-9, wherein the composition does not comprise ammonium sulfate.

12. The composition of any one of claims 1-11, further comprising L histidine at a concentration from about 0.1% (w/v) to about 0.5% (w/v) of the liquid component.

13. The composition of any one of claims 1-11, wherein the composition does not comprise L histidine.

14. The composition of any one of claims 1-13, further comprising lactose monohydrate at a concentration from about 0.1% (w/v) to about 0.5% (w/v) of the liquid component.

15. The composition of any one of claims 1-13, wherein the composition does not comprise lactose monohydrate.

16. The composition of any one of claims 1-15, wherein the composition does not comprise sucrose.

17. The composition of any one of claims 1-16, wherein the composition does not comprise dextrose.

18. The composition of any one of claims 1-4, wherein the composition consists essentially of the lipid particle, the cyclodextrin, HEPES, and water.

19. The composition of any one of claims 1-18, wherein the peptide conjugate is a platelet binding peptide (PBP) conjugate, a von Willebrand factor-binding peptide (VBP) conjugate, or a collagen-binding peptide (CBP) conjugate, or a combination thereof, wherein the PBP conjugate is a fibrinogen mimetic peptide (FMP) conjugate or a P-selectin binding peptide conjugate, and wherein the plurality of PBP conjugates, VBP conjugates, and/or CBP conjugates are conjugated to an outer surface of the lipid particle, wherein the PBP conjugates, VBP conjugates, and CBP conjugates, taken together, are present at <5 molar percent of the lipid particle.

20. A composition comprising a plurality of lipid particles and a liquid component, wherein the liquid component comprises water and a lyoprotectant at a concentration from about 1% (w/v) to about 20% (w/v) of the liquid component, wherein each lipid particle comprises a lipid and a plurality of peptide conjugates; the peptide conjugates are selected from platelet binding peptide (PBP) conjugates, von Willebrand factor-binding peptide (VBP) conjugates, and collagen-binding peptide (CBP) conjugates, or a combination thereof, wherein the PBP conjugate is a fibrinogen mimetic peptide (FMP) conjugate or a P-selectin binding peptide conjugate; and the plurality of PBP conjugates, VBP conjugates, and/or CBP conjugates are conjugated to an outer surface of the particle, wherein the PBP conjugates, VBP conjugates, and CBP conjugates, taken together, are present at <5 molar percent of the particle.

21. The composition of claim 20, wherein the lyoprotectant is cyclodextrin, sucrose, dextrose, or trehalose, or a combination thereof.

22. The composition of claim 20, wherein the lyoprotectant is a cyclodextrin.

23. The composition of claim 20, wherein the lyoprotectant is hydroxypropyl-β-cyclodextrin (HP-β-CD), hydroxypropyl-γ-cyclodextrin, hydroxypropyl-α-cyclodextrin, and/or hydroxypropyl cyclodextrin.

24. The composition of claim 22, wherein the cyclodextrin is hydroxypropyl-β-cyclodextrin (HP-β-CD).

25. The composition of any one of claims 20-24, further comprising 4-(2-hydroxyethyl)-1-piperazine ethane sulfonic acid (HEPES) at a concentration from about 0.1% (w/v) to about 0.5% (w/v) of the lipid component, wherein the pH of the liquid component is from about 5 to about 9.

26. The composition of any one of claims 20-24, wherein the composition does not comprise 4-(2-hydroxyethyl)-1-piperazine ethane sulfonic acid (HEPES).

27. The composition of any one of claims 20-26, further comprising tris(hydroxymethyl)aminomethane (tris) at a concentration from about 0.1% (w/v) to about 0.5% (w/v) of the liquid component.

28. The composition of any one of claims 20-26, wherein the composition does not comprise tris(hydroxymethyl)aminomethane (tris).

29. The composition of any one of claims 20-28, further comprising sodium chloride at a concentration from about 0.10% (w/v) to about 1% (w/v) of the liquid component.

30. The composition of any one of claims 20-28, wherein the composition does not comprise sodium chloride.

31. The composition of any one of claims 20-30, further comprising ammonium sulfate at a concentration from about 0.11% (w/v) to about 0.5% (w/v) of the liquid component.

32. The composition of any one of claims 20-30, wherein the composition does not comprise ammonium sulfate.

33. The composition of any one of claims 20-32, further comprising L histidine at a concentration from about 0.1% (w/v) to about 0.5% (w/v) of the liquid component.

34. The composition of any one of claims 20-32, wherein the composition does not comprise L histidine.

35. The composition of any one of claims 20-34, further comprising lactose monohydrate at a concentration from about 0.1% (w/v) to about 0.5% (w/v) of the liquid component.

36. The composition of any one of claims 20-34, wherein the composition does not comprise lactose monohydrate.

37. The composition of any one of claims 20-35, wherein the composition does not comprise sucrose.

38. The composition of any one of claims 20-36, wherein the composition does not comprise dextrose.

39. The composition of any one of claims 20-25, wherein the composition consists essentially of the lipid particle, the cyclodextrin, HEPES, and water.

40. The composition of claim 19 or claim 20, wherein the PBPs, VBPs, and/or CBPs or combination thereof, are spatially or topographically arranged on the lipid particle surface such that the PBPs, VBPs, and/or CBPs or combinations thereof, do not spatially mask each other.

41. The composition of any one of claims 19-40, wherein the PBPs, VBPs, and/or CBPs or combination thereof, are conjugated to the lipid particle surface with PEG linkers.

42. The composition of any one of claims 19-41, wherein the lipid particle comprises DSPE-PEG2 k-PBP, DSPE-PEG2 k-VBP, or DSPE-PEG2 k-CBP.

43. The composition of any one of claims 19-42, wherein the PBP conjugate comprises DSPE-PEG2 k-FMP, wherein the FMP is of Formula (I):

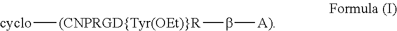

44. The composition of any one of claims 19-43, wherein the PBP conjugate comprises the amino acid sequence of SEQ ID NO:2.

45. The composition of any one of claims 19-44, wherein the PBP conjugate comprises the amino acid sequence of SEQ ID NO:3 or the amino acid sequence of SEQ ID NO:4.

46. The composition of any one of claims 19-45, wherein the PBP conjugate comprises the amino acid sequence of SEQ ID NO:5.

47. The composition of any one of claims 19-46, wherein the PBP is flanked by additional amino acid sequences.

48. The composition of any one of claims 19-47, wherein the VBPs comprise SEQ ID NO: 6.

49. The composition of claim 48, wherein the VBP is flanked by additional amino acid sequences.

50. The composition of any one of claims 19-49, wherein the CBPs comprise SEQ ID NO: 7.

51. The composition of claim 50, wherein the CBP is flanked by additional amino acid sequences.

52. The composition of any one of claims 19-51, wherein the lipid particle comprises the plurality of PBP conjugates, a plurality of VBP conjugates, and a plurality of CBP conjugates.

53. The composition of any one of claims 19-52, wherein the molar percentage of PBP conjugate is between 0.05% and 5.00% molar percent of the lipid particle.

54. The composition of any one of claims 19-52, wherein the molar percentage of FMP conjugate is between 0.05% and 5.00% molar percent of the lipid particle.

55. The composition of any one of claims 19-54, wherein the molar percentage of CBP conjugate is between 0.10% and 1.50% molar percent of lipid particle.

56. The composition of any one of claims 19-55, wherein the molar percentage of VBP conjugate is between 0.10% and 1.50% molar percent of the lipid particle.

57. The composition of any one of claims 19-56, wherein the molar percentage of PBP conjugate is about 1.00% of the lipid particle, the molar percentage of CBP conjugate is about 0.50% of the lipid particle, and the molar percentage of VBP conjugate is about 0.50% of the lipid particle.

58. The composition of any one of claims 19-57, wherein the lipid particle comprises both VBP conjugates and CBP conjugates; and the ratio of VPBs to CBPs provided on the lipid particle surface is about 70:30 to about 30:70.

59. The composition of any one of claims 19-58, wherein the relative molar ratios of PBP:CBP:VBP are 2:1:1.

60. The composition of any one of claims 19-56, wherein the relative molar ratios of PBP:CBP:VBP are 1:5:5.

61. The composition of any one of claims 19-56, wherein the relative molar ratios of PBP:CBP:VBP are 10:5:1.

62. The composition of any one of claims 19-56, wherein the relative molar ratios of PBP:CBP:VBP are 10:1:5.

63. The composition of any one of claims 19-56, wherein the relative molar ratios of PBP:CBP:VBP are 1:1:1.

64. The composition of any one of claims 19-56, wherein the relative molar ratios of PBP:CBP:VBP are 1:2:1.

65. The composition of any one of claims 19-56, wherein the relative molar ratios of PBP:CBP:VBP are 1:1:2.

66. The composition of any one of claims 19-56, wherein the relative molar ratios of PBP:CBP:VBP are 10:1:1.

67. The composition of any one of claims 19-57, wherein no CBP and/or VBP is conjugated to the surface of the lipid particle.

68. The composition of any one of claims 19-47, wherein the relative molar ratios of PBP:CBP are 2:1.

69. The composition of any one of claims 19-47, wherein the relative molar ratios of PBP:VBP are 2:1.

70. The composition of any one of claims 1-69, wherein the lipid comprises a phospholipid.

71. The composition of any one of claims 1-69, wherein the lipid comprises a phospholipid conjugated to polyethylene glycol (PEG).

72. The composition of claim 71, wherein the PEG conjugated to the phospholipid has an average molecular weight from about 500 Da to about 5500 Da.

73. The composition of any one of claims 1-72, wherein the lipid comprises a cholesterol, distearoylphosphatidylcholine (DSPC), or 1,2-distearoyl-sn-glycero-3-phosphoethanolamine (DSPE).

74. The composition of any one of claims 1-73, wherein the lipid particle has a diameter of about 1-1000 nm.

75. The composition of any one of claims 1-74, wherein the lipid particle has a diameter of about 30-300 nm.

76. The composition of any one of claims 1-75 in the form of a liposome.

77. The composition of any one of claims 1-76, wherein the lipid particle is substantially spherical.

78. The composition of any one of claims 1-77, wherein the lipid particle has a polydispersity index less than or equal to 0.3.

79. The composition of any one of claims 1-78, wherein the lipid particle has a net positive or net negative zeta potential.

80. The composition of any one of claims 1-79, wherein the particle promotes platelet aggregation.

81. The composition of any one of claims 1-80, the particle binds to activated platelets.

82. The composition of any one of claims 1-81, wherein the particle binds to ADP- or TRAP-activated platelets.

83. A method of forming a dried composition, comprising:
incubating a composition of any one of claims 1-82 at a temperature from about 0 to about −210° C. to form a cooled composition; and
incubating the cooled composition at a temperature from about 0 to about −60° C. at a pressure from about 0.1 to about 1.0 mTorr, thereby forming a dried composition.

84. The method of claim 83, further comprising cooling a composition of any one of claims 1-46 at a rate between −1.0° C./second and −0.1° C./second before incubating the composition.

85. The method of claim 83 or 84, wherein the lipid particles in the dried composition have a diameter from about 1 nm to about 1000 nm.

86. The method of any one of claims 83-85, wherein the lipid particles in the dried composition have an average diameter from about 1 nm to about 1000 nm.

87. The method of any one of claims 83-86, wherein the lipid particles in the dried composition have a spherical morphology.

88. The method of any one of claims 83-87, wherein the lipid particles in the dried composition have a polydispersity index of no greater than 0.3.

89. The method of any one of claims 83-88, wherein the lipid particles in the dried composition have a net positive or net negative zeta potential.

90. The method of any one of claims 83-89, wherein the lipid particles in the dried composition have a shelf-life of at least 2 years at ambient conditions.

91. The method of any one of claims 83-90, wherein the lipid particles in the dried composition have a shelf-life of at least 2 years at storage conditions between −20° C. to 4° C.

92. The method of any one of claims 83-91, wherein the lipid particles in the dried composition have a shelf-life of at least 2 years at storage conditions up to 50° C.

93. The method of any one of claims 83-92, wherein the lipid particles in the dried composition retain loading of one or more therapeutic agents.

94. A composition made by a method of any one of claims 83-93.

95. A method of forming a reconstituted composition comprising contacting a composition of claim 94 with water, for example, for less than about 30 seconds, thereby forming the reconstituted composition.

96. The method of claim 95, wherein the reconstituted composition comprises lipid particles having a diameter from about 1 nm to about 1000 nm.

97. The method of claim 96, wherein the lipid particles have an average diameter from about 30 nm to about 300 nm.

98. The method of claim 96 or claim 97, wherein the lipid particles have a substantially spherical morphology.

99. The method of any one of claims 96-98, wherein the lipid particles have a polydispersity index of no greater than 0.3.

100. A reconstituted composition made by a method of any one of claims 95-99.

101. A method of diminishing bleeding, treating hemorrhage, treating a vascular injury, promoting hemostasis, preventing or inhibiting platelet aggregation, promoting aggregation of activated platelets on a site with exposed vWF and collagen, or treating cancer in a subject in need thereof, comprising administering to the subject an effective amount of a reconstituted composition of claim 100.

102. The method of claim 101, wherein the reconstituted composition is administered intravenously.

103. The method of claim 101 or claim 102, wherein the reconstituted composition is injected as a bolus or into a normal saline or dextrose bag and administered intravenously.

104. The method of claim 101, wherein the reconstituted composition is aerosolized and administered through respiration, for example, orally or intranasally or both.

105. The method of claim 101, wherein the reconstituted composition is administered intraosseously.

106. The method of claim 101, wherein the reconstituted composition is administered transdermally.

107. The method of any one of claims 101-106, wherein the method is a method of treating hemorrhage, and the hemorrhage is a noncompressible hemorrhage, for example, resulting from trauma.

108. The method of any one of claims 101-106, wherein the method is a method of diminishing bleeding, wherein the bleeding is due to surgery.

109. The method of any one of claims 101-106, wherein the method is a method of diminishing bleeding, wherein the bleeding is due to thrombocytopenia, postpartum hemorrhage, traumatic brain injury, von Willebrand disease, Glanzmann thrombasthenia, or Bernard Soulier syndrome.


SEQUENCE LISTING (9 sequences)

SEQ ID NO: 1 - protein, 9 aa, synthetic construct
  SITE 7: note = Tyrosine ethyl ester
  SITE 9: note = Beta-alanine
CNPRGDYRX

SEQ ID NO: 2 - protein, 9 aa, synthetic construct
  SITE 1: note = Propargyl modified residue
  SITE 7: note = Tyrosine ethyl ester
  SITE 1..9: note = Cyclized via an intrachain disulfide bond
CNPRGDYRC

SEQ ID NO: 3 - protein, 6 aa, synthetic construct
GRGDSP

SEQ ID NO: 4 - protein, 12 aa, synthetic construct
HHLGGAKQAGDV

SEQ ID NO: 5 - protein, 8 aa, synthetic construct
DAEWVDVS

SEQ ID NO: 6 - protein, 15 aa, synthetic construct
TRYLRIHPQSWVHQI

SEQ ID NO: 7 - protein, 21 aa, synthetic construct
  SITE 3: note = Hydroxyproline
  SITE 6: note = Hydroxyproline
  SITE 9: note = Hydroxyproline
  SITE 12: note = Hydroxyproline
  SITE 15: note = Hydroxyproline
  SITE 18: note = Hydroxyproline
  SITE 21: note = Hydroxyproline
GPXGPXGPXGPXGPXGPXGPX

SEQ ID NO: 8 - protein, 21 aa, synthetic construct
  SITE 3: note = Hydroxyproline
  SITE 6: note = Hydroxyproline
  SITE 9: note = Hydroxyproline
  SITE 12: note = Hydroxyproline
  SITE 15: note = Hydroxyproline
  SITE 18: note = Hydroxyproline
  SITE 21: note = Hydroxyproline
GPXGPXGPXGPXGPXGPXGPX

SEQ ID NO: 9 - protein, 5 aa, synthetic construct
EWVDV